(19) United States
(12) Patent Application Publication
(10) Pub. No.: US 2023/0416460 A1
(43) Pub. Date: Dec. 28, 2023

(54) COMPOUND, RESIN COMPOSITION, RESIN SHEET, RESIN CURED PRODUCT, AND LAMINATED SUBSTRATE

(71) Applicant: TDK CORPORATION, Tokyo (JP)

(72) Inventors: Takashi INAGAKI, Tokyo (JP); Hiroshi ITO, Tokyo (JP)

(73) Assignee: TDK CORPORATION, Tokyo (JP)

(21) Appl. No.: 18/035,192

(22) PCT Filed: Feb. 25, 2022
(86) PCT No.: PCT/JP2022/008037

§ 371 Date: May 3, 2023

(30) Foreign Application Priority Data
Mar. 31, 2021 (JP) 2021-061845

(51) Int. Cl. C08G 65/38

(52) U.S. Cl. CPC C08G 65/38

(57) ABSTRACT

A compound having a chain structure consisting of an aromatic ring group, an ether oxygen atom, a methylene group, an aromatic ring group, a methylene group, an ether oxygen atom, and an aromatic ring group bonded in an order, wherein a hydroxymethyl group is bonded to a carbon atom of the aromatic ring group disposed at a first end of the chain structure, and one end group selected from a hydroxyl group, an amino group, an amide group, and a carboxyl group is bonded to a carbon atom of the aromatic ring group disposed at a second end of the chain structure.

### TECHNICAL FIELD

The present invention relates to a compound, a resin composition, a resin sheet, a resin cured product, and a laminated substrate.

The present application claims priority on the basis of Japanese Patent Application No. 2021-061845, filed Mar. 31, 2021, the content of which is incorporated herein by reference.

### BACKGROUND ART

In recent years, along with the demand for the miniaturization of electronic devices, mounting of electronic components has been with higher functionality and higher density. Therefore, the handling of heat generated from electronic components has become important.

The heat generated from electronic components is dissipated to the outside mainly through a substrate. A power supply laminated substrate obtained by laminating resin substrates is required to have, in particular, high heat dissipation properties. Therefore, inorganic particles such as alumina, boron nitride, and magnesium oxide are added to the resin substrates to enhance the thermal conductivity of the resin substrates. For example, Patent Document 1 describes an epoxy resin composition containing an epoxy resin, a curing agent, and an inorganic filler.

However, when the content ratio of the inorganic particles in the resin substrate is increased in order to improve the thermal conductivity, the processability and strength of the resin substrate are reduced. Accordingly, in order to obtain a resin substrate having high heat dissipation properties even if the content ratio of the inorganic particles in the resin substrate is reduced to ensure the processability and strength of the resin substrate, the development of a resin, from which a cured product having high thermal conductivity can be obtained, is underway.

As the resin having high thermal conductivity, an epoxy resin into which a mesogenic skeleton is introduced has been mentioned (see, for example, Non Patent Document 1).

Moreover, Patent Document 2 discloses a mixture of epoxy resins obtained by reacting at least a bifunctional epoxy resin with a biphenol compound.

Patent Document 3 discloses a resin composition that contains a filler and a thermosetting resin having a mesogenic group in the molecule.

### CITATION LIST

### Patent Document

### [Patent Document 1]

Japanese Patent Application No. 6074447

### [Patent Document 2]

Japanese Unexamined Patent Application, First Publication No. 2012-131992

### [Patent Document 3]

PCT International Publication No. WO 2013/065159

### Non Patent Document

### [Non Patent Document 1]

Yutaka TAKEZAWA, Polymer, Vol. 65, February issue, pp. 65 to 67, 2016

### SUMMARY OF INVENTION

### Technical Problem

However, resin compositions in the related art are not capable of producing cured products having sufficiently high thermal conductivity.

The present invention has been completed in consideration of the aforementioned problems, and an object of the present invention is to provide a compound used as a material for a resin composition from which a cured product having high thermal conductivity is obtained.

Moreover, another object of the present invention is to provide: a resin composition which contains the compound according to the present invention and from which a cured product having high thermal conductivity is obtained; a resin sheet, a resin cured product, and a laminated substrate.

### Solution to Problem

In order to achieve the objects, the present inventors focused on the skeleton and end groups of the compound used as the material for the resin composition, and repeated intensive studies.

As a result, the present inventors have found that a compound which has a chain structure obtained by bonding an aromatic ring group which may have a substituent, an ether oxygen atom, and a methylene group in a specific order, and in which a hydroxymethyl group is bonded to a carbon atom of the aromatic ring group disposed at a first end of the chain structure, and one end group selected from a hydroxyl group, an amino group, an amide group, and a carboxyl group is bonded to a carbon atom of the aromatic ring group disposed at a second end of the chain structure, is preferable.

That is, the present invention relates to the following inventions.

[1] A compound having a chain structure consisting of an aromatic ring group, an ether oxygen atom, a methylene group, an aromatic ring group, a methylene group, an ether oxygen atom, and an aromatic ring group bonded in an order,
wherein a hydroxymethyl group is bonded to a carbon atom of the aromatic ring group disposed at a first end of the chain structure, and
one end group selected from a hydroxyl group, an amino group, an amide group, and a carboxyl group is bonded to a carbon atom of the aromatic ring group disposed at a second end of the chain structure.

[2] The compound as described in [1], including:
a first aromatic ring unit composed of a first aromatic ring group and two ether oxygen atoms bonded to the first aromatic ring group;
a second aromatic ring unit composed of a second aromatic ring group and two methylene groups bonded to the second aromatic ring group;
a third aromatic ring unit composed of a third aromatic ring group and a hydroxymethyl group bonded to the third aromatic ring group; and
a fourth aromatic ring unit composed of a fourth aromatic ring group and the end group bonded to the fourth aromatic ring group,
in which the chain structure has a skeleton in which the first aromatic ring unit and the second aromatic ring unit are alternately disposed and the first aromatic ring unit is disposed at both ends, the third aromatic ring group is bonded to a first end of the skeleton via a methylene group, and the fourth aromatic ring group is bonded to a second end of the skeleton via a methylene group, or
the chain structure has a skeleton in which the first aromatic ring unit and the second aromatic ring unit are alternately disposed and the second aromatic ring unit is disposed at both ends, the third aromatic ring group is bonded to a first end of the skeleton via an ether oxygen atom, and the fourth aromatic ring group is bonded to a second end of the skeleton via an ether oxygen atom.

[3] The compound as described in [1], including:
a first aromatic ring unit composed of a first aromatic ring group and two ether oxygen atoms bonded to the first aromatic ring group;
a second aromatic ring unit composed of a second aromatic ring group and two methylene groups bonded to the second aromatic ring group;
a third aromatic ring unit composed of a third aromatic ring group and a hydroxymethyl group bonded to the third aromatic ring group; and
a fourth aromatic ring unit composed of a fourth aromatic ring group and the end group bonded to the fourth aromatic ring group,
in which the chain structure has a skeleton in which the first aromatic ring unit and the second aromatic ring unit are alternately disposed, and
the third aromatic ring group is bonded to an end on the first aromatic ring unit side via a methylene group, and the fourth aromatic ring group is bonded to an end on the second aromatic ring unit side via an ether oxygen atom, or
the fourth aromatic ring group is bonded to an end on the first aromatic ring unit side via a methylene group, and the third aromatic ring group is bonded to an end on the second aromatic ring unit side via an ether oxygen atom.

[4] The compound as described in [1], which is represented by one of General Formulae (1) to (4).

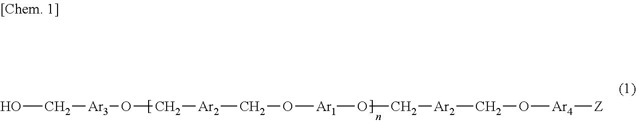

(In Formula (1), Ar₁'s are each independently a first aromatic ring group which may have a substituent, Ar₂'s are each independently a second aromatic ring group which may have a substituent, Ar₃ is a third aromatic ring group which may have a substituent, and Ar₄ is a fourth aromatic ring group which may have a substituent. Z is the end group. n is an integer of 0 or more.)

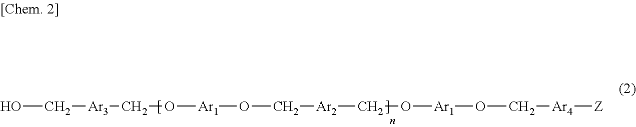

(In Formula (2), Ar₁'s are each independently a first aromatic ring group which may have a substituent, Ar₂'s are each independently a second aromatic ring group which may have a substituent, Ar₃ is a third aromatic ring group which may have a substituent, and Ar₄ is a fourth aromatic ring group which may have a substituent. Z is the end group. n is an integer of 1 or more.)

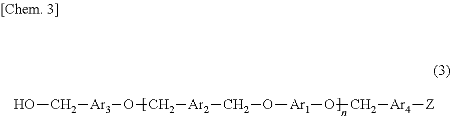

(In Formula (3), Ar₁'s are each independently a first aromatic ring group which may have a substituent, Ar₂'s are each independently a second aromatic ring group which may have a substituent, Ar₃ is a third aromatic ring group which may have a substituent, and Ar₄ is a fourth aromatic ring group which may have a substituent. Z is the end group. n is an integer of 1 or more.)

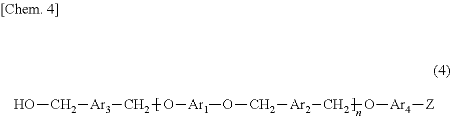

(In Formula (4), Ar₁'s are each independently a first aromatic ring group which may have a substituent, Ar₂'s are each independently a second aromatic ring group which may have a substituent, Ar₃ is a third aromatic ring group which may have a substituent, and Ar₄ is a fourth aromatic ring group which may have a substituent. Z is the end group. n is an integer of 1 or more.)

[5] The compound as described in any one of [2] to [4], in which one or more of the first aromatic ring group, the second aromatic ring group, the third aromatic ring group, and the fourth aromatic ring group are any aromatic ring groups represented by Formulae (5) to (9).

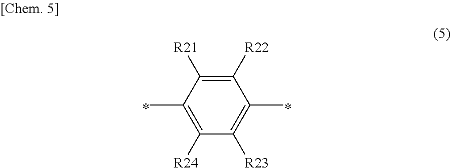

(In Formula (5), R21 to R24 are each independently one selected from hydrogen, a methyl group, a trifluoromethyl group, a halogen group, a nitro group, and a cyano group. * is a bond.)

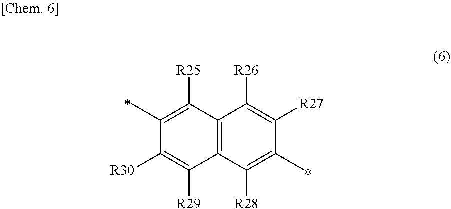

(In Formula (6), R25 to R30 are each independently one selected from hydrogen, a methyl group, a trifluoromethyl group, a halogen group, a nitro group, and a cyano group. * is a bond.)

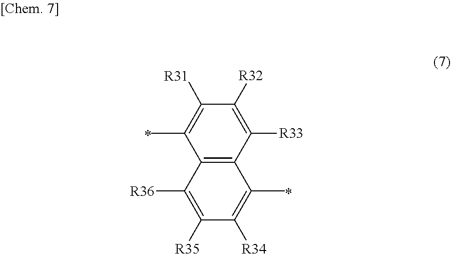

(In Formula (7), R31 to R36 are each independently one selected from hydrogen, a methyl group, a trifluoromethyl group, a halogen group, a nitro group, and a cyano group. * is a bond.)

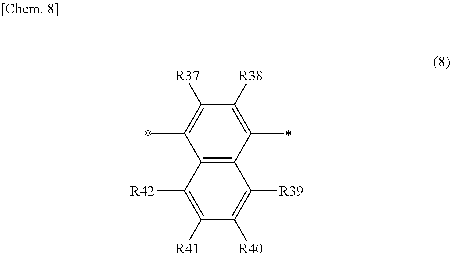

(In Formula (8), R37 to R42 are each independently one selected from hydrogen, a methyl group, a trifluoromethyl group, a halogen group, a nitro group, and a cyano group. * is a bond.)

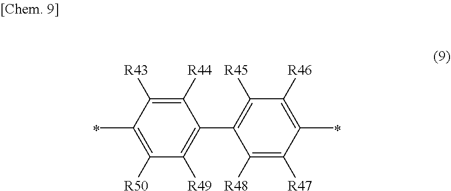

(In Formula (9), R43 to R50 are each independently one selected from hydrogen, a methyl group, a trifluoromethyl group, a halogen group, a nitro group, and a cyano group. * is a bond.)

[6] The compound as described in any one of [2] to [5], in which one or more of the first aromatic ring group, the second aromatic ring group, the third aromatic ring group, and the fourth aromatic ring group are paraphenylene groups which may have a substituent.

[7] The compound as described in any one of [2] to [5],
in which the first aromatic ring group and the fourth aromatic ring group are a same, and
the second aromatic ring group is a paraphenylene group.

[8] The compound as described in [1], which is represented by one of General Formulae (10) to (13).

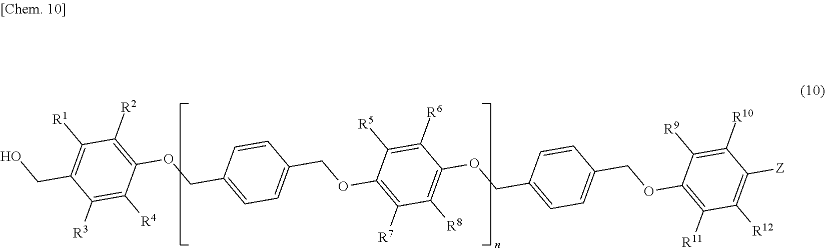

(In Formula (10), R1 to R12 are each independently one selected from hydrogen, a methyl group, a trifluoromethyl group, a halogen group, a nitro group, and a cyano group. Z is the end group. n is an integer of 1 or more.)

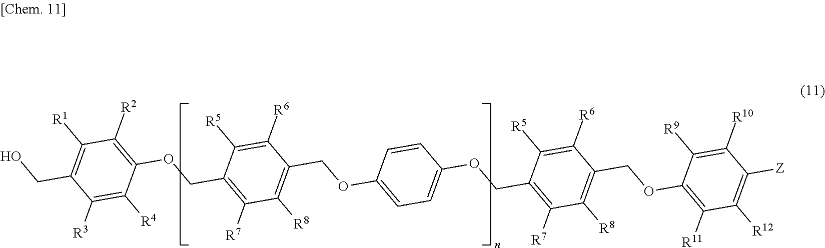

(In Formula (11), R1 to R12 are each independently one selected from hydrogen, a methyl group, a trifluoromethyl group, a halogen group, a nitro group, and a cyano group. Z is the end group. n is an integer of 1 or more.)

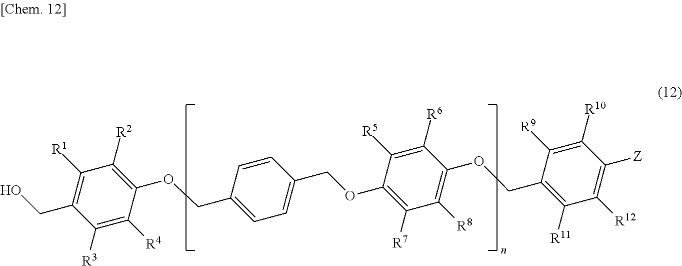

(In Formula (12), R1 to R12 are each independently one selected from hydrogen, a methyl group, a trifluoromethyl group, a halogen group, a nitro group, and a cyano group. Z is the end group. n is an integer of 1 or more.)

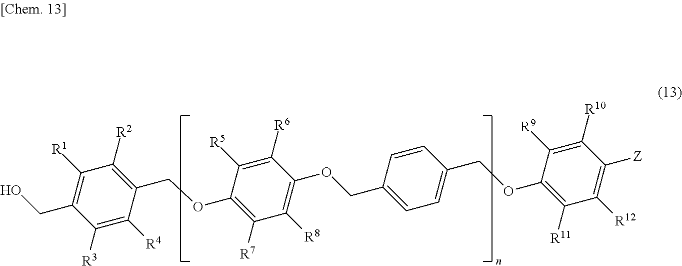

(In Formula (13), R1 to R12 are each independently one selected from hydrogen, a methyl group, a trifluoromethyl group, a halogen group, a nitro group, and a cyano group. Z is the end group. n is an integer of 1 or more.)

[9] The compound as described in [8], which is the compound represented by General Formula (13),
in which R1 to R4 are each hydrogen,
one among R5 to R8 is a methyl group, and the others are each hydrogen, and
one among R9 to R12 is a methyl group, and the others are each hydrogen.

[10] A resin composition containing the compound as described in any one of [1] to [9].

[11] The resin composition as described in [10], which has a chain structure consisting of an aromatic ring group, an ether oxygen atom, a methylene group, an aromatic ring group, a methylene group, an ether oxygen atom, and an aromatic ring group bonded in an order, the resin composition comprising one or both of a compound in which one end group selected from a hydroxyl group, an amino group, an amide group, and a carboxyl group is bonded to a carbon atom of each of the aromatic ring groups disposed at both ends of the chain structure, and a compound in which a hydroxymethyl group is bonded to a carbon atom of each of the aromatic ring groups disposed at both ends of the chain structure.

[12] A resin composition containing an epoxy resin and a curing agent,
in which the curing agent includes the compound as described in any one of [1] to [9].

[13] A resin sheet containing the resin composition as described in any one of [10] to [12].

[14] A resin cured product which includes a cured product of the resin composition as described in any one of [10] to [12].

[15] A laminated substrate obtained by laminating a plurality of resin substrates, in which at least one among the plurality of resin substrates is the resin cured product as described in [14].

### Advantageous Effects of Invention

The compound according to the present invention has a chain structure obtained by bonding an aromatic ring group, an ether oxygen atom, a methylene group, an aromatic ring group, a methylene group, an ether oxygen atom, and an aromatic ring group in this order. The chain structure included in the compound according to the present invention is a mesogenic group that exhibits liquid crystallinity, and has a structure in which an aromatic ring group that imparts rigidity and a methylene group and an ether oxygen atom that impart mobility are disposed in a specific order. For this reason, the compound according to the present invention can stabilize a smectic liquid crystal phase due to the moderate mobility of the mesogenic group itself. Therefore, the compound according to the present invention has high orientation properties.

Moreover, in the compound according to the present invention, a hydroxymethyl group is bonded to a carbon atom of the aromatic ring group disposed at a first end of the chain structure, and one end group selected from a hydroxyl group, an amino group, an amide group, and a carboxyl group is bonded to a carbon atom of the aromatic ring group disposed at a second end of the chain structure. Therefore, a cured product having a smectic liquid crystal structure resulting from a mesogenic structure and having high thermal conductivity is obtained by polymerizing a resin composition containing the compound according to the present invention.

### BRIEF DESCRIPTION OF DRAWINGS

FIG. 1 is a perspective view showing an example of the resin sheet and the resin substrate according to the present invention.

FIG. 2 is a cross-sectional view of the resin sheet and the resin substrate taken along line II-II shown in FIG. 1.

FIG. 3 is a perspective view showing an example of the laminated substrate according to the present invention.

FIG. 4 is a cross-sectional view of the laminated substrate taken along line IV-IV shown in FIG. 3.

### DESCRIPTION OF EMBODIMENTS

Hereinafter, the present invention will be described in detail with reference to the drawings, as appropriate. In the drawings used in the following description, the characteristic parts are shown enlarged in some cases for convenience in order to make it easier to understand the characteristics of the present invention. Accordingly, the dimensional ratios or the like of each constituent element shown in the drawings may be different from the actual one. The materials, dimensions, and the like exemplified in the following description are examples, and the present invention is not limited to the examples and can be appropriately modified and implemented within the scope in which the gist of the present invention is not modified.

### “Compound”

The compound according to the present embodiment has a chain structure obtained by bonding an aromatic ring group, an ether oxygen atom, a methylene group, an aromatic ring group, a methylene group, an ether oxygen atom, and an aromatic ring group in this order.

In the compound according to the present embodiment, a hydroxymethyl group is bonded to a carbon atom of the aromatic ring group disposed at a first end of the chain structure, and one end group selected from a hydroxyl group, an amino group, an amide group, and a carboxyl group is bonded to a carbon atom of the aromatic ring group disposed at a second end of the chain structure.

The compound according to the present embodiment preferably includes a first aromatic ring unit, a second aromatic ring unit, a third aromatic ring unit, and a fourth aromatic ring unit which are shown below.

The first aromatic ring unit is composed of a first aromatic ring group and two ether oxygen atoms bonded to the first aromatic ring group.

The second aromatic ring unit is composed of a second aromatic ring group and two methylene groups bonded to the second aromatic ring group.

The third aromatic ring unit is composed of a third aromatic ring group and a hydroxymethyl group bonded to the third aromatic ring group.

The fourth aromatic ring unit is composed of a fourth aromatic ring group and the end group bonded to the fourth aromatic ring group.

The chain structure in the compound according to the present embodiment may have a skeleton in which the first aromatic ring unit and the second aromatic ring unit are alternately disposed and the first aromatic ring unit is disposed at both ends. In this case, it is preferable that the third aromatic ring group be bonded to a first end of the skeleton via a methylene group, and the fourth aromatic ring group be bonded to a second end of the skeleton via a methylene group.

The chain structure in the compound according to the present embodiment may have a skeleton in which the first aromatic ring unit and the second aromatic ring unit are alternately disposed and the second aromatic ring unit is disposed at both ends. In this case, it is preferable that the third aromatic ring group be bonded to a first end of the skeleton via an ether oxygen atom, and the fourth aromatic ring group be bonded to a second end of the skeleton via an ether oxygen atom.

Since the chain structure has the skeleton in which the first aromatic ring unit and the second aromatic ring unit are alternately disposed and the first aromatic ring unit is disposed at both ends, or the skeleton in which the second aromatic ring unit is disposed at both ends, the chain structure is preferably considered to have a skeleton having a symmetrical structure. When the skeleton has a symmetrical structure, a cured product easily forms an ordered structure, and thus a cured product having even higher thermal conductivity is obtained.

The chain structure in the compound according to the present embodiment may have a skeleton in which the first aromatic ring unit and the second aromatic ring unit are alternately disposed, the first aromatic ring unit is disposed at one end, and the second aromatic ring unit is disposed at the other end.

When the chain structure has such a skeleton, it is preferable that the third aromatic ring group be bonded to an end on the first aromatic ring unit side via a methylene group, and the fourth aromatic ring group be bonded to an end on the second aromatic ring unit side via an ether oxygen atom. Moreover, it is preferable that the fourth aromatic ring group be bonded to an end on the first aromatic ring unit side via a methylene group, and the third aromatic ring group be bonded to an end on the second aromatic ring unit side via an ether oxygen atom.

The first aromatic ring group, the second aromatic ring group, the third aromatic ring group, and the fourth aromatic ring group in the compound according to the present embodiment may all be aromatic ring groups and may have a substituent. The first aromatic ring group, the second aromatic ring group, the third aromatic ring group, and the fourth aromatic ring group may be different from one another, or may be partially or entirely the same, and can be appropriately determined according to the use of the compound.

When the compound according to the present embodiment has a plurality of first aromatic ring groups, the plurality of first aromatic ring groups may be different from one another, or may be partially or entirely the same. A compound, in which all of the plurality of first aromatic ring groups are the same, can be easily produced and is thus preferable.

Moreover, when the compound according to the present embodiment has a plurality of second aromatic ring groups, the plurality of second aromatic ring groups may be different from one another, or may be partially or entirely the same. A compound, in which all of the plurality of second aromatic ring groups are the same, can be easily produced and is thus preferable.

In the compound according to the present embodiment, each of the substituents in the first aromatic ring group, the second aromatic ring group, the third aromatic ring group, and the fourth aromatic ring group is preferably one selected from a methyl group, a trifluoromethyl group, a halogen group, a nitro group, and a cyano group, can be appropriately determined according to the use of the compound, and is not particularly limited. Among these substituents, in particular, a methyl group, a trifluoromethyl group, and a halogen group are preferable from the viewpoint of chemical stability and environmental load reduction, and a methyl group is particularly preferable.

One or more of the first aromatic ring group, the second aromatic ring group, the third aromatic ring group, and the fourth aromatic ring group in the compound according to the present embodiment may be, for example, any aromatic ring groups represented by General Formulae (5) to (9). It is preferable that one or more of the first aromatic ring group, the second aromatic ring group, the third aromatic ring group, and the fourth aromatic ring group be aromatic ring groups represented by General Formulae (5) to (9), because in this case a polymer having higher thermal conductivity is obtained and a compound having favorable handleability is obtained.

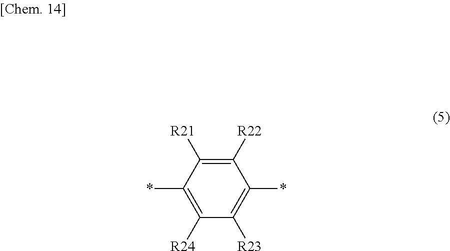

(In Formula (5), R21 to R24 are each independently one selected from hydrogen, a methyl group, a trifluoromethyl group, a halogen group, a nitro group, and a cyano group. * is a bond.)

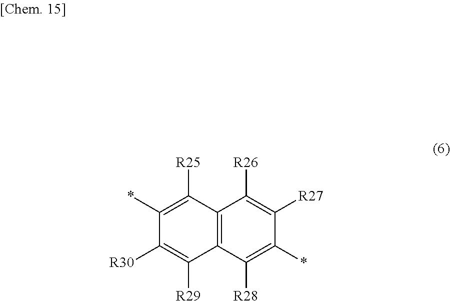

(In Formula (6), R25 to R30 are each independently one selected from hydrogen, a methyl group, a trifluoromethyl group, a halogen group, a nitro group, and a cyano group. * is a bond.)

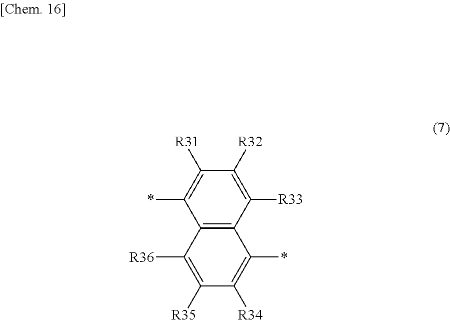

(In Formula (7), R31 to R36 are each independently one selected from hydrogen, a methyl group, a trifluoromethyl group, a halogen group, a nitro group, and a cyano group. * is a bond.)

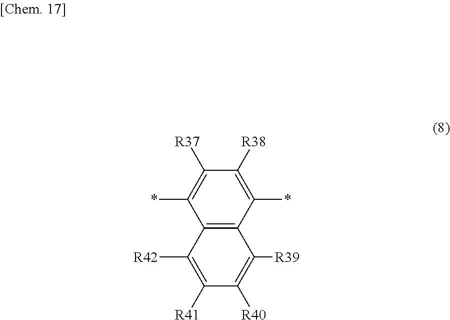

(In Formula (8), R37 to R42 are each independently one selected from hydrogen, a methyl group, a trifluoromethyl group, a halogen group, a nitro group, and a cyano group. * is a bond.)

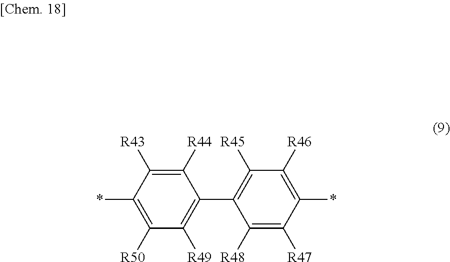

(In Formula (9), R43 to R50 are each independently one selected from hydrogen, a methyl group, a trifluoromethyl group, a halogen group, a nitro group, and a cyano group. * is a bond.)

One or more of the first aromatic ring group, the second aromatic ring group, the third aromatic ring group, and the fourth aromatic ring group in the compound according to the present embodiment are preferably phenylene groups which may have a substituent, in order to obtain a compound from which a polymer having higher thermal conductivity is obtained. The phenylene group in the phenylene groups which may have a substituent may be any of an orthophenylene group, a metaphenylene group, and a paraphenylene group, and is particularly preferably a paraphenylene group represented by Formula (5) because a compound having a skeleton exhibiting high orientation properties is obtained. In particular, it is preferable that the second aromatic ring group be a paraphenylene group, because in this case a compound including a structure in which methylene groups are bonded to both sides of the paraphenylene group is obtained, and a polymer having further favorable thermal conductivity is obtained.

In the compound according to the present embodiment, in particular, it is preferred that the first aromatic ring group and the fourth aromatic ring group be the same and the second aromatic ring group be a paraphenylene group. Such a compound easily takes a liquid crystal state and is thus advantageous for ordering.

Moreover, when the second aromatic ring group in the compound according to the present embodiment is a paraphenylene group that does not have a substituent, raw materials are easily available, and the compound having a low melting point and favorable solubility in a solvent is obtained.

Examples of the compound according to the present embodiment include compounds represented by General Formulae (1) to (4).

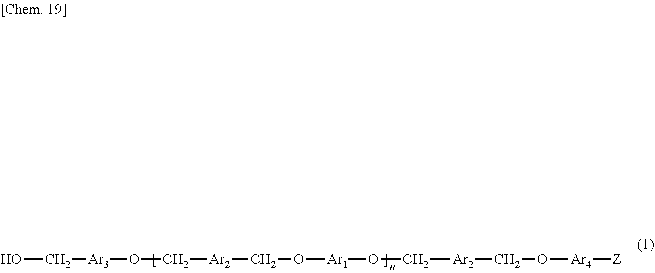

(In Formula (1), Ar₁'s are each independently a first aromatic ring group which may have a substituent, Ar₂'s are each independently a second aromatic ring group which may have a substituent, Ar₃ is a third aromatic ring group which may have a substituent, and Ar₄ is a fourth aromatic ring group which may have a substituent. Z is the end group. n is an integer of 0 or more.)

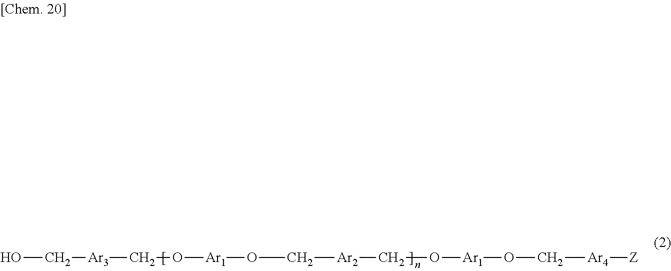

(In Formula (2), Ar₁'s are each independently a first aromatic ring group which may have a substituent, Ar₂'s are each independently a second aromatic ring group which may have a substituent, Ar₃ is a third aromatic ring group which may have a substituent, and Ar₄ is a fourth aromatic ring group which may have a substituent. Z is the end group. n is an integer of 1 or more.)

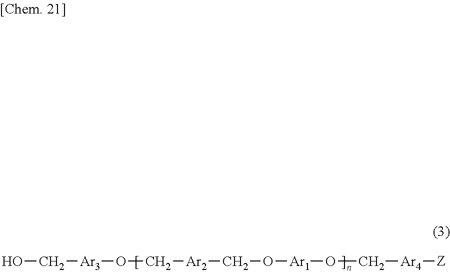

(In Formula (3), Ar₁'s are each independently a first aromatic ring group which may have a substituent, Ar₂'s are each independently a second aromatic ring group which may have a substituent, Ar₃ is a third aromatic ring group which may have a substituent, and Ar₄ is a fourth aromatic ring group which may have a substituent. Z is the end group. n is an integer of 1 or more.)

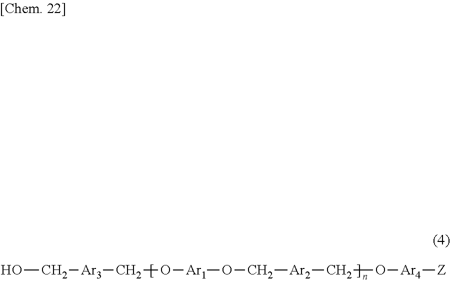

(In Formula (4), Ar₁'s are each independently a first aromatic ring group which may have a substituent, Ar₂'s are each independently a second aromatic ring group which may have a substituent, Ar₃ is a third aromatic ring group which may have a substituent, and Ar₄ is a fourth aromatic ring group which may have a substituent. Z is the end group. n is an integer of 1 or more.)

The compounds represented by General Formulae (1) to (4) all include a first aromatic ring unit (represented by —O—Ar₁—O— in Formulae (1) to (4)), a second aromatic ring unit (represented by —CH₂—Ar₁—CH₂— in Formulae (1) to (4)), a third aromatic ring unit (represented by —Ar₃—CH₂—OH in Formulae (1) to (4)), and a fourth aromatic ring unit (represented by —Ar₄—Z in Formulae (1) to (4)).

In the compounds represented by General Formulae (1) to (4), the first aromatic ring unit has the first aromatic ring group (represented by Ar₁ in Formulae (1) to (4)) and two ether oxygen atoms bonded to the first aromatic ring group.

The second aromatic ring unit has the second aromatic ring group (represented by Ar₂ in Formulae (1) to (4)) and two methylene groups bonded to the second aromatic ring group.

The third aromatic ring unit is composed of the third aromatic ring group (represented by Ar₃ in Formulae (1) to (4)) and a hydroxymethyl group (represented by —CH₂—OH in Formulae (1) to (4)).

The fourth aromatic ring unit is composed of the fourth aromatic ring group (represented by Ar₄ in Formulae (1) to (4)) and an end group (represented by Z in Formulae (1) to (4)).

The compound represented by General Formula (1) has a skeleton in which the first aromatic ring unit and the second aromatic ring unit are alternately disposed in a chain shape, and both ends are terminated with the second aromatic ring units. In the compound represented by General Formula (1), the methylene groups of the second aromatic ring unit are disposed at both ends of the skeleton, the second aromatic ring unit at a first end is bonded to the third aromatic ring group represented by Ar₃ in Formula (1) via an ether oxygen atom, and the second aromatic ring unit at a second end is bonded to the fourth aromatic ring group represented by Ar₄ in Formula (1) via an ether oxygen atom.

Moreover, the compound represented by General Formula (2) has a skeleton in which the first aromatic ring unit and the second aromatic ring unit are alternately disposed in a chain shape, and both ends are terminated with the first aromatic ring units. In the compound represented by General Formula (2), the ether oxygen atoms of the first aromatic ring unit are disposed at both ends of the skeleton, the first aromatic ring unit at a first end is bonded to the third aromatic ring group represented by Ar₃ in Formula (2) via a methylene group, and the first aromatic ring unit at a second end is bonded to the fourth aromatic ring group represented by Ar₄ in Formula (1) via methylene oxygen.

Further, the compound represented by General Formula (3) has a skeleton in which the first aromatic ring unit and the second aromatic ring unit are alternately disposed in a chain shape. In the compound represented by General Formula (3), the fourth aromatic ring group is bonded to an end on the first aromatic ring unit side via a methylene group, and the third aromatic ring group is bonded to an end on the second aromatic ring unit side via an ether oxygen atom.

Furthermore, the compound represented by General Formula (4) has a skeleton in which the first aromatic ring unit and the second aromatic ring unit are alternately disposed in a chain shape. In the compound represented by General Formula (4), the third aromatic ring group is bonded to an end on the first aromatic ring unit side via a methylene group, and the fourth aromatic ring group is bonded to an end on the second aromatic ring unit side via an ether oxygen atom.

Accordingly, the compounds represented by General Formulae (1) to (4) are all a compound in which a hydroxymethyl group (—CH₂—OH) is bonded to the carbon atom of the aromatic ring group disposed at the first end of the chain structure, and an end group represented by Z in Formulae (1) to (4) is bonded to the carbon atom of the aromatic ring group disposed at the second end of the chain structure.

In the compound according to the present embodiment, examples of a compound, in which the first aromatic ring group, the third aromatic ring group, and the fourth aromatic ring group are paraphenylene groups which may have a substituent and is represented by Formula (5), and the second aromatic ring group is a paraphenylene group which does not have a substituent, include compounds represented by General Formulae (10) to (13).

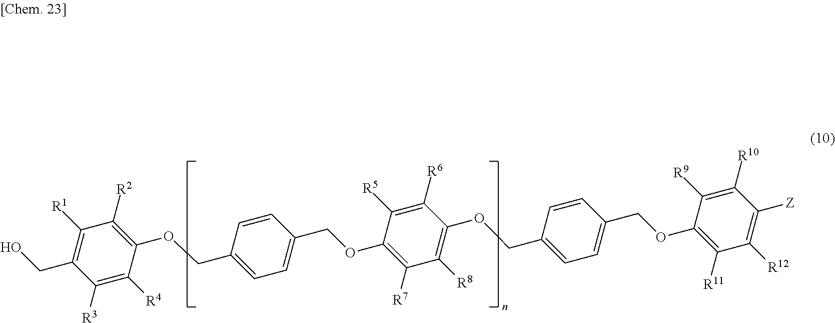

(In Formula (10), R1 to R12 are each independently one selected from hydrogen, a methyl group, a trifluoromethyl group, a halogen group, a nitro group, and a cyano group. Z is the end group. n is an integer of 1 or more.)

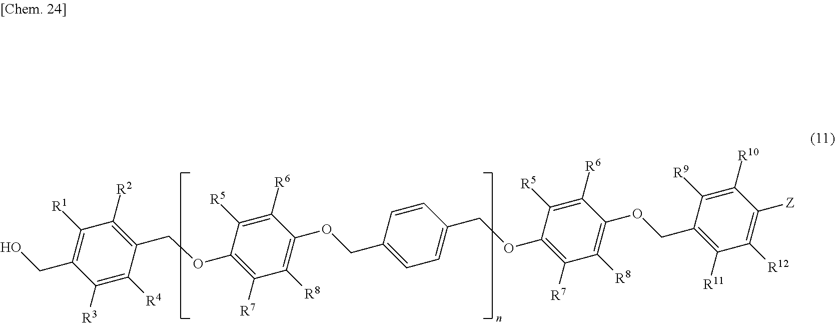

(In Formula (11), R1 to R12 are each independently one selected from hydrogen, a methyl group, a trifluoromethyl group, a halogen group, a nitro group, and a cyano group. Z is the end group. n is an integer of 1 or more.)

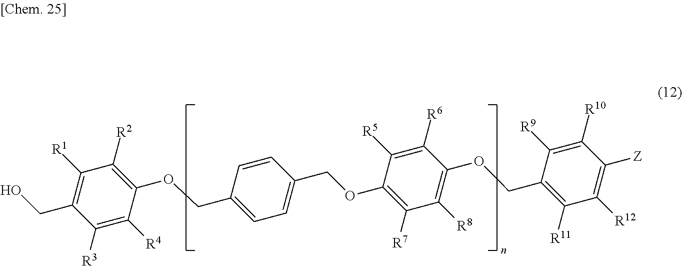

(In Formula (12), R1 to R12 are each independently one selected from hydrogen, a methyl group, a trifluoromethyl group, a halogen group, a nitro group, and a cyano group. Z is the end group. n is an integer of 1 or more.)

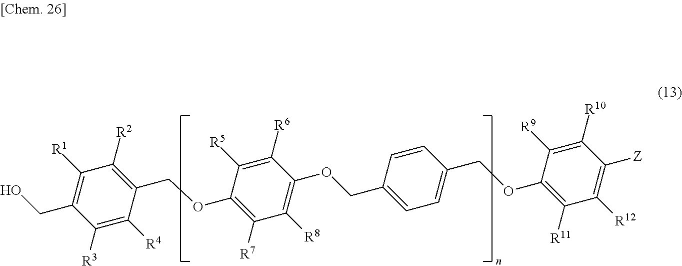

(In Formula (13), R1 to R12 are each independently one selected from hydrogen, a methyl group, a trifluoromethyl group, a halogen group, a nitro group, and a cyano group. Z is the end group. n is an integer of 1 or more.)

In the compounds represented by General Formulae (10) to (13), the first aromatic ring group, the third aromatic ring group, and the fourth aromatic ring group are paraphenylene groups which may have a substituent and are represented by Formula (5), and the second aromatic ring group is a paraphenylene group which does not have a substituent. Accordingly, the compounds represented by General Formulae (10) to (13) have a skeleton including a structure, in which methylene groups are bonded to both sides of a paraphenylene group which does not have a substituent, and thus exhibit high orientation properties. Therefore, a resin composition containing the compounds represented by General Formulae (10) to (13) provides a polymer having further favorable thermal conductivity. Moreover, in the compounds represented by General Formulae (10) to (13), the second aromatic ring group is a paraphenylene group which does not have a substituent, and thus the raw materials are easily available.

The compound represented by General Formula (10) has a skeleton having a symmetrical structure composed of the first aromatic ring unit and the second aromatic ring unit, a cured product easily forms an ordered structure, and thus a cured product having even higher thermal conductivity is obtained. Moreover, since the compound represented by General Formula (10) has an asymmetric molecular structure in which the structure of a first end of the skeleton is different from the structure of a second end, a curing reaction proceeds step by step. That is, in the compound represented by General Formula (10), the curing reaction can be controlled by appropriately selecting the type of end group according to the use or the like. Furthermore, the compound represented by General Formula (10) has excellent solubility because a hydroxymethyl group is bonded to the carbon atom of the aromatic ring group disposed at the first end of the chain structure.

The compound represented by General Formula (11) has a skeleton having a symmetrical structure composed of the first aromatic ring unit and the second aromatic ring unit, a cured product easily forms an ordered structure, and thus a cured product having even higher thermal conductivity is obtained. Moreover, since the compound represented by General Formula (11) has an asymmetric molecular structure in which the structure of a first end of the skeleton is different from the structure of a second end, a curing reaction proceeds step by step. That is, in the compound represented by General Formula (11), the curing reaction can be controlled by appropriately selecting the type of end group according to the use or the like. Furthermore, the compound represented by General Formula (11) has excellent solubility because a hydroxymethyl group is bonded to the carbon atom of the aromatic ring group disposed at the first end of the chain structure.

The compound represented by General Formula (12) has a skeleton in which the first aromatic ring unit and the second aromatic ring unit are alternately disposed, a cured product easily forms an ordered structure, and thus a cured product having even higher thermal conductivity is obtained. Moreover, since the compound represented by General Formula (12) has an asymmetric molecular structure in which the structure of a first end of the skeleton is different from the structure of a second end, a curing reaction proceeds step by step. That is, in the compound represented by General Formula (12), the curing reaction can be controlled by appropriately selecting the type of end group according to the use or the like. Furthermore, the compound represented by General Formula (12) has excellent solubility because a hydroxymethyl group is bonded to the carbon atom of the aromatic ring group disposed at the first end of the chain structure.

The compound represented by General Formula (13) has a skeleton in which the first aromatic ring unit and the second aromatic ring unit are alternately disposed, a cured product easily forms an ordered structure, and thus a cured product having even higher thermal conductivity is obtained. Moreover, since the compound represented by General Formula (13) has an asymmetric molecular structure in which the structure of a first end of the skeleton is different from the structure of a second end, a curing reaction proceeds step by step. That is, in the compound represented by General Formula (13), the curing reaction can be controlled by appropriately selecting the type of end group according to the use or the like. Furthermore, the compound represented by General Formula (13) has excellent solubility because a hydroxymethyl group is bonded to the carbon atom of the aromatic ring group disposed at the first end of the chain structure.

When the compound according to the present embodiment is the compound represented by General Formula (13), it is preferable that, in Formula (13), R1 to R4 be each hydrogen, one among R5 to R8 be a methyl group and the others be each hydrogen, and one among R9 to R12 be a methyl group and the others be each hydrogen. The raw materials of such a compound are easily available, and such a compound can be easily produced.

In the compound according to the present embodiment, the first aromatic ring group and the second aromatic ring group may be the same as or different from each other. Accordingly, both the first aromatic ring group and the second aromatic ring group may be paraphenylene groups which do not have a substituent. In this case, the raw materials are easily available, which is preferable. Moreover, when the first aromatic ring group and the second aromatic ring group are different from each other, the symmetry of the structure in the skeleton is lower than when the first aromatic ring group and the second aromatic ring group are the same. Therefore, the crystallinity of the compound is reduced, and a smectic liquid crystal phase is stabilized. As a result, a compound, from which a polymer having further favorable thermal conductivity is obtained, is obtained.

In the compounds represented by General Formulae (1) to (4) and (10) to (13), n is the number of repeating units described in parentheses. In the compound represented by General Formula (1), n is an integer of 0 or more. When n is 0 or more, the effect of improving the thermal conductivity of the polymer due to having the aforementioned skeleton is achieved. n is preferably 1 or more and more preferably 2 or more so that the effect of improving the thermal conductivity of the polymer due to having the aforementioned skeleton becomes more remarkable. In the compounds represented by General Formulae (2) to (4) and (10) to (13), n is an integer of 1 or more. When n is 1 or more, the effect of improving the thermal conductivity of the polymer due to having the aforementioned skeleton is achieved. n is preferably 2 or more so that the effect of improving the thermal conductivity of the polymer due to having the aforementioned skeleton becomes more remarkable.

Furthermore, the upper limit of n in General Formulae (1) to (4) and (10) to (13) is not particularly limited, but is preferably 20 or less in order to ensure the solubility of the compound in a solvent. n is more preferably 10 or less and still more preferably 6 or less because a compound having further favorable solubility in a solvent is obtained.

The skeleton of the compound according to the present embodiment preferably has a repeating unit composed of one first aromatic ring unit and one second aromatic ring unit. The compound according to the present embodiment may be a mixture containing a plurality of types of compounds having different numbers of repeating units, or may be a single type of compound having the same number of repeating units.

When the compound according to the present embodiment is a mixture containing a plurality of types of compounds having different numbers of repeating units, the average degree of polymerization, which is the average number of repeating units of the compound contained in the mixture, is preferably 1.0 to 6.0 and more preferably 2.0 to 5.0. When the average degree of polymerization is 1.0 or greater, a resin composition containing the compound provides a polymer having even higher thermal conductivity. Moreover, when the average degree of polymerization is 6.0 or less, a compound having further favorable solubility in a solvent is obtained.

In the compound according to the present embodiment, a hydroxymethyl group is bonded to the carbon atom of the third aromatic ring group disposed at the first end of the chain structure. Moreover, one end group selected from a hydroxyl group, an amino group, an amide group, and a carboxyl group is bonded to the carbon atom of the fourth aromatic ring group disposed at the second end of the chain structure. The end group can be appropriately determined according to the use of the compound or the like.

In the compound according to the present embodiment, one end group selected from a hydroxyl group, an amino group, an amide group, and a carboxyl group can be easily bonded to the carbon atom of the fourth aromatic ring group disposed at the second end of the chain structure. Accordingly, the compound according to the present embodiment can be easily produced. In particular, it is preferable that the end group be a hydroxyl group, because in this case the compound can be efficiently produced with a small number of steps. Moreover, these end groups react with epoxy groups. Therefore, the compound according to the present embodiment can be suitably used as a curing agent for an epoxy resin.

In addition, a polymerization product (cured product), which is obtained by polymerizing a resin composition containing the compound according to the present embodiment and an epoxy resin, has high thermal conductivity. When the compound according to the present embodiment is used as a curing agent for the epoxy resin, the end group is particularly preferably a hydroxyl group among the aforementioned end groups. This is because a resin composition containing a compound, in which a hydroxymethyl group is bonded to a first end and a hydroxyl group as the end group is bonded to a second end, and an epoxy resin facilitates the control of a polymerization reaction and provides a cured product having further favorable thermal conductivity and chemical stability.

In the compound according to the present embodiment, the type of end group can be appropriately selected according to the use or the like. Doing so can adjust the reactivity with other monomers and the like in a resin composition containing the compound according to the present embodiment.

### “Method for Producing Compound”

The compound according to the present embodiment can be produced, for example, by the following method. In the present embodiment, methods for producing compounds represented by General Formulae (1) to (4) will be described as examples of the method for producing the compound.

### “Method for Producing Compound Represented by General Formula (1)”

A first raw material, which is an aromatic compound having two phenolic hydroxyl groups, and a second raw material, which is an aromatic compound having a monohalogenated methyl group, are prepared.

Moreover, the first raw material and the second raw material are subjected to a bimolecular nucleophilic substitution reaction (S_{N}2 reaction) using potassium carbonate to synthesize a first precursor compound having a skeleton from which the chain structure in the compound according to the present embodiment is derived. At this time, by increasing the molar ratio of the second raw material to the first raw material, a first precursor compound having a skeleton, in which structures derived from the second raw material are disposed at both ends, is produced. Conditions for reacting the first raw material with the second raw material can be appropriately determined according to a combination of the first raw material and the second raw material, and are not particularly limited.

The first raw material used in the method for producing a compound according to the present embodiment is an aromatic compound having two phenolic hydroxyl groups, and is appropriately selected according to the structure of the first aromatic ring group in the produced compound. Examples of the first raw material include methylhydroquinone, hydroquinone, tetramethylhydroquinone, trimethylhydroquinone, 2-(trifluoromethyl)-1,4-benzenediol, fluorohydroquinone, chlorohydroquinone, bromohydroquinone, 2,5-dihydroxynitrobenzene, tetrafluorohydroquinone, tetrachlorohydroquinone, tetrabromohydroquinone, 2,6-dihydroxynaphthalene, 1,5-dihydroxynaphthalene, 4,4′-dihydroxybiphenyl, and 3,3′,5,5′-tetramethylbiphenyl-4,4′-diol.

The second raw material used in the method for producing a compound according to the present embodiment is an aromatic compound having a monohalogenated methyl group, and is appropriately selected according to the structure of the second aromatic ring group in the produced compound. Examples of the second raw material include α,α′-dichloro-p-xylene, 1,4-bis(chloromethyl)-2-methylbenzene, 3,6-bis(chloromethyl)durene, 1,4-bis(bromomethyl)-2-fluorobenzene, 1,4-bis(bromomethyl)-2-chlorobenzene, 2-bromo-1,4-bis(bromomethyl)benzene, 1,4-bis(chloromethyl)-2-nitrobenzene, 1,4-bis(bromomethyl)-2,3,5,6-tetrafluorobenzene, α,α′,2,3,5,6-hexachloro-p-xylene, 1,2,4,5-tetrabromo-3,6-bis(bromomethyl)benzene, 1,2-dibromo-3,6-bis(chloromethyl)-4,5-dimethylbenzene, 1,4-bis(bromomethyl)-2,5-dimethylbenzene, 4,4′-bis(chloromethyl)biphenyl, 2,6-bis(bromomethyl)naphthalene, 1,5-bis(chloromethyl)naphthalene, and 1,4-bis(bromomethyl)naphthalene.

Next, the first precursor compound is reacted with a third raw material, which is an aromatic compound having a hydroxymethyl group to synthesize a second precursor compound. Conditions for reacting the first precursor compound with the third raw material can be appropriately determined according to a combination of the first precursor compound and the third raw material, and are not particularly limited.

The third raw material used in the method for producing a compound according to the present embodiment is an aromatic compound having a hydroxymethyl group, and is appropriately selected according to the structure of the third aromatic ring group or the like in the produced compound.

When the first precursor compound has a structure in which the elements disposed at both ends of the skeleton are derived from the second raw material, specific examples of the third raw material include 2,6-difluoro-4-hydroxy-benzyl alcohol, 2-fluoro-4-hydroxy-benzyl alcohol, 3-bromo-4-hydroxy-benzyl alcohol, 4-hydroxy-3-nitro-benzyl alcohol, 4-hydroxy-3-methyl-benzyl alcohol, 4-hydroxy-3,5-dimethyl-benzyl alcohol, 2-chloro-4-hydroxy-benzyl alcohol, 3,5-difluoro-4-hydroxy-benzyl alcohol, 4-hydroxy-2,6-dimethyl-benzyl alcohol, 4-hydroxy-2-methyl-benzyl alcohol, 4-hydroxy-2-nitro-benzyl alcohol, 4-hydroxy-3-(trifluoromethyl)-benzyl alcohol, and 4-hydroxy-2,5-dimethyl-benzyl alcohol.

Next, the second precursor compound obtained by reacting the first precursor compound with the third raw material is reacted with a fourth raw material, which is an aromatic compound having a structure from which an end group is derived, to obtain a compound represented by General Formula (1).

Conditions for reacting the second precursor compound with the fourth raw material can be appropriately determined according to a combination of the second precursor compound and the fourth raw material, and are not particularly limited.

The fourth raw material used in the method for producing a compound according to the present embodiment is an aromatic compound having a structure from which an end group is derived, and is appropriately selected according to the structure of the fourth aromatic ring group, the structure of the end group, or the like in the produced compound.

In the present embodiment, the first precursor compound has a structure in which the elements disposed at both ends of the skeleton are derived from the second raw material, and thus an aromatic compound having two phenolic hydroxyl groups can be used as the fourth raw material, like the first raw material. Moreover, an aromatic compound having one phenolic hydroxyl group and an amino group or a carboxyalkyl group may be used as the fourth raw material. Specific examples of the fourth raw material include methylhydroquinone, hydroquinone, 2-fluoro-1,4-benzenediol, 2,3,5,6-tetrafluoro-1,4-benzenediol, 2,3-difluoro-1,4-benzenediol, methyl 4-hydroxybenzoate, and p-aminophenol.

In the present embodiment, when the end group of the compound represented by General Formula (1), which is obtained by the aforementioned method, is a carboxyl group, a compound represented by Formula (1), in which the end group is an amide group (—CONH₂), may be produced by amidating the end group, for example, through the reaction between the compound represented by General Formula (1) and aqueous ammonia.

“Method for producing compound represented by General Formula (2)” Similarly to the method for producing a compound represented by General Formula (1), a first raw material, which is an aromatic compound having two phenolic hydroxyl groups, and a second raw material, which is an aromatic compound having a monohalogenated methyl group, are prepared.

Moreover, similarly to the method for producing a compound represented by General Formula (1), the first raw material and the second raw material are subjected to a bimolecular nucleophilic substitution reaction (S_{N}2 reaction) using potassium carbonate to synthesize a first precursor compound having a skeleton from which the chain structure in the compound according to the present embodiment is derived.

When producing a compound represented by General Formula (2), by reducing the molar ratio of the second raw material to the first raw material, a first precursor compound having a skeleton, in which structures derived from the first raw material are disposed at both ends, is produced.

As the first raw material and the second raw material used when producing a compound represented by General Formula (2), the same raw materials as in the method for producing a compound represented by General Formula (1) can be used.

Next, the first precursor compound is reacted with a fourth raw material, which is an aromatic compound having a structure from which an end group is derived, to synthesize a second precursor compound. Conditions for reacting the first precursor compound with the fourth raw material can be appropriately determined according to a combination of the first precursor compound and the fourth raw material, and are not particularly limited.

The fourth raw material used in the method for producing a compound according to the present embodiment is an aromatic compound having a structure from which an end group is derived, and is appropriately selected according to the structure of the fourth aromatic ring group or the like in the produced compound.

When the first precursor compound has a structure in which the elements disposed at both ends of the skeleton are derived from the first raw material, it is preferable to use an aromatic compound having a monohalogenated methyl group, as the fourth raw material. Specific examples of the fourth raw material include methyl 4-(bromomethyl)benzoate, methyl 4-(bromomethyl)-3-fluoro-benzoate, methyl 2-bromo-4-(bromomethyl)benzoate, methyl 4-(bromomethyl)-3-chloro-benzoate, methyl 4-(chloromethyl)-3,5-difluoro-benzoate, methyl 4-(bromomethyl)-2-methyl-benzoate, methyl 4-(bromomethyl)-3-nitro-benzoate, methyl 2-chloro-4-(chloromethyl)-6-cyano-benzoate, methyl 4-(bromomethyl)-2,6-difluoro-benzoate, methyl 4-(bromomethyl)-3-trifluoromethyl-benzoate, methyl 4-(bromomethyl)-2,5-difluoro-benzoate, methyl 4-(bromomethyl)-3-cyano-benzoate, methyl 5-bromo-4-(bromomethyl)-2-fluoro-benzoate, methyl 4-(bromomethyl)-2-nitro-benzoate, methyl 4-(bromomethyl)-2,3-difluoro-benzoate, 1-(bromomethyl)-4-nitro-benzene, 1-(bromomethyl)-4-nitro-2-(trifluoromethyl)-benzene, and 4-(bromomethyl)-2-methyl-1-nitro-benzene.

Next, the second precursor compound obtained by reacting the first precursor compound with the fourth raw material is reacted with a third raw material, which is an aromatic compound having a hydroxymethyl group, to synthesize a third precursor compound. Conditions for reacting the second precursor compound with the third raw material can be appropriately determined according to a combination of the second precursor compound and the third raw material, and are not particularly limited.

The third raw material used in the method for producing a compound according to the present embodiment is an aromatic compound having a hydroxymethyl group, and is appropriately selected according to the structure of the third aromatic ring group, the structure of the end group, or the like in the produced compound.

When producing a compound represented by General Formula (2), specific examples of the third raw material include 5-(bromomethyl)-2-hydroxymethyl-2-benzonitrile.

Thereafter, the third precursor compound is reacted with a compound having a structure, from which an end group represented by Z in Formula (2) is derived, to obtain a compound represented by General Formula (2).

In the present embodiment, when the end group of the compound represented by General Formula (2), which is obtained by the aforementioned method, is a carboxyl group, a compound represented by Formula (2), in which the end group is an amide group (—CONH₂), may be produced by amidating the end group, for example, through the reaction between the compound represented by General Formula (2) and aqueous ammonia.

### “Method for Producing Compound Represented by General Formula (3)”

Similarly to the methods for producing compounds represented by General Formula (1) and General Formula (2), a first raw material, which is an aromatic compound having two phenolic hydroxyl groups, and a second raw material, which is an aromatic compound having a monohalogenated methyl group, are prepared.

Moreover, similarly to the methods for producing compounds represented by General Formula (1) and General Formula (2), the first raw material and the second raw material are subjected to a bimolecular nucleophilic substitution reaction (S_{N}2 reaction) using potassium carbonate to synthesize a first precursor compound having a skeleton from which the chain structure in the compound according to the present embodiment is derived.

When producing a compound represented by General Formula (3), by making the molar ratio of the first raw material and the second raw material approximately 1, a first precursor compound having a skeleton, in which a structure derived from the first raw material is disposed at one end and a structure derived from the second raw material is disposed at the other end, is produced.

As the first raw material and the second raw material used when producing a compound represented by General Formula (3), the same raw materials as in the methods for producing compounds represented by General Formula (1) and General Formula (2) can be used.

Thereafter, as needed, the first precursor compound is reacted with a compound having a structure, from which an end group represented by Z in Formula (3) is derived, to obtain a compound represented by General Formula (3).

When the first aromatic ring group and the fourth aromatic ring group are the same, the second aromatic ring group and the third aromatic ring group are the same, and an end group in the compound represented by General Formula (3) is a hydroxyl group, the first precursor compound becomes the compound represented by General Formula (3) according to the present embodiment.

### “Method for Producing Compound Represented by General Formula (4)”

Similarly to the methods for producing compounds represented by General Formula (1) to General Formula (3), a first raw material, which is an aromatic compound having two phenolic hydroxyl groups, and a second raw material, which is an aromatic compound having a monohalogenated methyl group, are prepared.

Moreover, similarly to the methods for producing compounds represented by General Formula (1) to General Formula (3), the first raw material and the second raw material are subjected to a bimolecular nucleophilic substitution reaction (S_{N}2 reaction) using potassium carbonate to synthesize a first precursor compound having a skeleton from which the chain structure in the compound according to the present embodiment is derived.

When producing a compound represented by General Formula (4), by increasing the molar ratio of the second raw material to the first raw material similarly to the method for producing a compound represented by General Formula (1), a first precursor compound having a skeleton, in which structures derived from the second raw material are disposed at both ends, is produced.

As the first raw material and the second raw material used when producing a compound represented by General Formula (4), the same raw materials as in the methods for producing compounds represented by General Formula (1) to General Formula (3) can be used.

Next, the first precursor compound is reacted with a fourth raw material, which is an aromatic compound having a structure from which an end group is derived, to synthesize a second precursor compound. Conditions for reacting the first precursor compound with the fourth raw material can be appropriately determined according to a combination of the first precursor compound and the fourth raw material, and are not particularly limited.

The fourth raw material used in the method for producing a compound according to the present embodiment is an aromatic compound having a structure from which an end group is derived, and is appropriately selected according to the structure of the fourth aromatic ring group or the like in the produced compound.

When producing a compound represented by General Formula (4), the first precursor compound has a structure in which the elements disposed at both ends of the skeleton are derived from the second raw material, and thus the same raw material as in the method for producing a compound represented by General Formula (1) can be used as the fourth raw material.

Thereafter, a hydroxymethyl group is introduced into the aromatic ring group, which serves as the third aromatic ring group in Formula (4), in a second precursor compound to obtain a compound represented by General Formula (4).

In the present embodiment, when the end group of the compound represented by General Formula (4), which is obtained by the aforementioned method, is a carboxyl group, a compound represented by Formula (4), in which the end group is an amide group (—CONH₂), may be produced by amidating the end group, for example, through the reaction between the compound represented by General Formula (4) and aqueous ammonia.

In the method for producing a compound according to the present embodiment, it is preferable to produce a compound in which the end group bonded to the carbon atom of the aromatic ring group disposed at the first end and/or the second end of the chain structure is different from that of the compound according to the present embodiment, simultaneously with the compound according to the present embodiment.

When producing a polymer using a resin composition containing the compound according to the present embodiment, it is preferable to mix and use a compound different from the compound according to the present embodiment, according to needs such as the use, in some cases. In the case where a compound, in which the end group bonded to the carbon atom of the aromatic ring group disposed at the first end and/or the second end of the chain structure is different from that of the compound according to the present embodiment, is produced simultaneously with the compound according to the present embodiment, when producing a resin composition containing the compound according to the present embodiment, the resin composition can be efficiently produced in some cases without performing a step of mixing a plurality of types of compounds.

In the method for producing a compound according to the present embodiment, a compound, in which the end group bonded to the carbon atom of the aromatic ring group disposed at the first end and/or the second end of the chain structure is different from that of the compound according to the present embodiment, is produced simultaneously with the compound according to the present embodiment to obtain a mixture, and then a specific single type of compound may be separated from the mixture using a known method, as needed.

The compound according to the present embodiment has a chain structure obtained by bonding an aromatic ring group, an ether oxygen atom, a methylene group, an aromatic ring group, a methylene group, an ether oxygen atom, and an aromatic ring group in this order. The chain structure is a mesogenic group that exhibits liquid crystallinity, and has a structure in which an aromatic ring group that imparts rigidity and a methylene group and an ether oxygen atom that impart mobility are disposed in a specific order.

In addition, in the compound according to the present embodiment, a hydroxymethyl group is bonded to the carbon atom of the aromatic ring group disposed at the first end of the chain structure, and one end group selected from a hydroxyl group, an amino group, an amide group, and a carboxyl group is bonded to the carbon atom of the aromatic ring group disposed at the second end of the chain structure. Accordingly, a cured product, which has a smectic liquid crystal structure resulting from a mesogenic structure of the compound and has high thermal conductivity and suppressed phonon scattering, is obtained by polymerizing a resin composition containing the compound according to the present embodiment. Moreover, the compound according to the present embodiment has excellent solubility because a hydroxymethyl group is bonded to the carbon atom of the aromatic ring group disposed at the first end of the chain structure.

### “Resin Composition”

The resin composition according to the present embodiment contains the aforementioned compound according to the present embodiment. The resin composition according to the present embodiment may contain only one type of the compound according to the present embodiment, or may contain two or more types thereof.

It is preferable that the resin composition according to the present embodiment contain other components as needed, in addition to the compound according to the present embodiment.

Examples of the other components include one or more types of compounds having a chain structure obtained by bonding an aromatic ring group, an ether oxygen atom, a methylene group, an aromatic ring group, a methylene group, an ether oxygen atom, and an aromatic ring group in this order, like the compound according to the present embodiment. As such compounds, for example, a compound in which one end group selected from a hydroxyl group, an amino group, an amide group, and a carboxyl group is bonded to the carbon atom of each of the aromatic ring groups disposed at both ends of the aforementioned chain structure, and a compound in which a hydroxymethyl group is bonded to the carbon atom of each of the aromatic ring groups disposed at both ends of the aforementioned chain structure are mentioned.

The resin composition according to the present embodiment may contain an epoxy resin and the compound according to the present embodiment. In this case, the compound according to the present embodiment functions as a curing agent for the epoxy resin.

As the epoxy resin, for example, known epoxy compounds, such as 4,4′-biphenol diglycidyl ether, 3,3′,5,5′-tetramethyl-4,4′-bis(glycidyloxy)-1,1′-biphenyl, triglycidyl isocyanurate, triglycidyl-p-aminophenol, 1,6-bis(2,3-epoxypropan-1-yloxy)naphthalene, a cresol novolac epoxy resin, a novolac epoxy resin, a bisphenol A-type epoxy resin, a bisphenol F-type epoxy resin, a naphthalene-type epoxy resin, and a tetraglycidyldiaminodiphenylmethane-type epoxy resin, can be used, and a commercially available epoxy resin may be used. Only one kind of the epoxy resin may be contained, or two or more kinds thereof may be contained.

The resin composition according to the present embodiment may contain other resin components as needed, in addition to the compound according to the present embodiment and the epoxy resin. Examples of the other resin components include an amino group-containing compound such as p-phenylenediamine, an amide group-containing compound such as sulfanilamide, and a compound such as a phenolic resin. Only one kind of the other resin components may be contained, or two or more kinds thereof may be contained.

The resin composition according to the present embodiment may contain a curing accelerator as needed, in addition to the compound according to the present embodiment. For example, when the resin composition according to the present embodiment contains the epoxy resin and the compound according to the present embodiment, a basic organic compound having a high boiling point or the like can be used as the curing accelerator. Specific examples of the curing accelerator include those having a boiling point of 200° C. or higher and selected from tertiary amines, tertiary phosphines, 4-dimethylaminopyridine (DMAP), and imidazoles. Among them, it is preferable to use 2-ethyl-4-methylimidazole (2E4MZ) and 1-(2-cyanoethyl)-2-phenylimidazole, which are imidazole-based epoxy resin curing accelerators, as the curing accelerator, in particular, from the viewpoint of ease of handling.

The resin composition according to the present embodiment may contain a curing agent as needed, in addition to the compound according to the present embodiment. Examples of the curing agent include p-phenylenediamine, 1,5-diaminonaphthalene, hydroquinone, 2,6-dihydroxynaphthalene, phloroglucinol, 4-hydroxybenzoic acid, 6-hydroxy-2-naphthoic acid, 4-aminobenzoic acid, phenolic resin, and polyamidoamine. Among them, it is particularly preferable to use 4-aminobenzoic acid as the curing agent because in this case a cured product having higher thermal conductivity is obtained.

The resin composition according to the present embodiment may contain inorganic particles as needed. Examples of the inorganic particles include boron nitride particles, magnesium oxide particles, alumina particles, aluminum hydroxide particles, aluminum nitride particles, and silica particles. Only one kind of the inorganic particles may be contained alone, or two or more kinds thereof may be contained.

The content of the inorganic particles is preferably 200 to 700 parts by mass and more preferably 300 to 600 parts by mass with respect to 100 parts by mass of the total of the resin composition components other than the inorganic particles. When the content of the inorganic particles is 200 parts by mass or greater, the effect of improving the thermal conductivity of a cured product of the resin composition becomes remarkable. Moreover, when the content of the inorganic particles is 700 parts by mass or less, sufficient molding processability is obtained when molding a resin substrate using a cured product of the resin composition.

The resin composition according to the present embodiment may contain a solvent as needed. Examples of the solvent include ketones such as acetone and methyl ethyl ketone (MEK), alcohols such as methanol, ethanol, and isopropanol, aromatic compounds such as toluene and xylene, ethers such as tetrahydrofuran (THF) and 1,3-dioxolane, esters such as ethyl acetate and γ-butyrolactone, and amides such as N,N-dimethylformamide (DMF) and N-methylpyrrolidone. Only one kind of the solvent may be used alone, or two or more kinds thereof may be used in combination.

The resin composition according to the present embodiment may contain optional components other than the aforementioned components, as needed. Examples of the optional components include a coupling agent such as a silane coupling agent or a titanate coupling agent, a flame retardant such as halogen, a plasticizer, and a lubricant.

The resin composition according to the present embodiment can be produced, for example, by a method for mixing the aforementioned compound according to the present embodiment with the other components that are contained as needed.

Since the resin composition according to the present embodiment contains the aforementioned compound according to the present embodiment, a polymer (cured product) having high thermal conductivity is obtained by polymerizing the resin composition.

### “Resin Sheet”

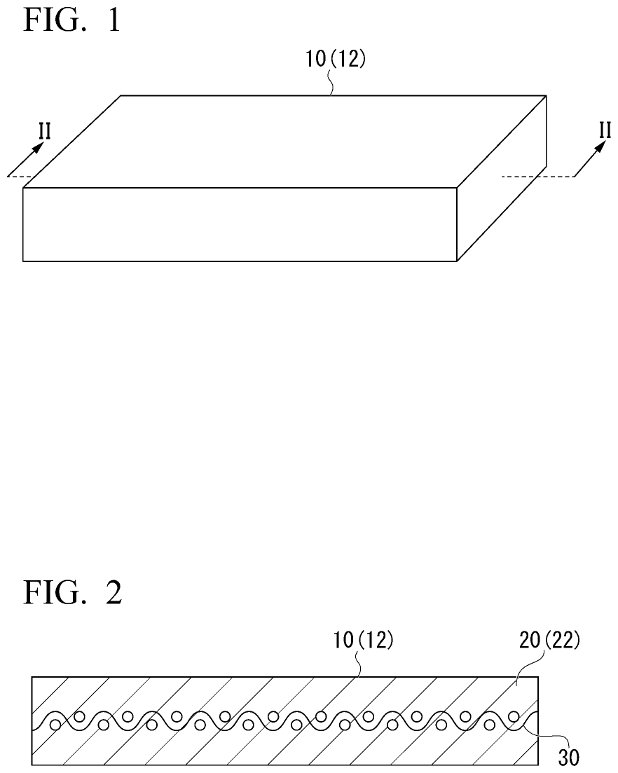

FIG. 1 is a perspective view showing an example of the resin sheet and the resin substrate according to the present invention. A resin sheet 12 shown in FIG. 1 is a sheet obtained by molding the resin composition according to the present embodiment. The resin sheet 12 may contain the resin composition as it is, or may contain the resin composition of which a part or the whole is in a B-stage (semi-cured) state.

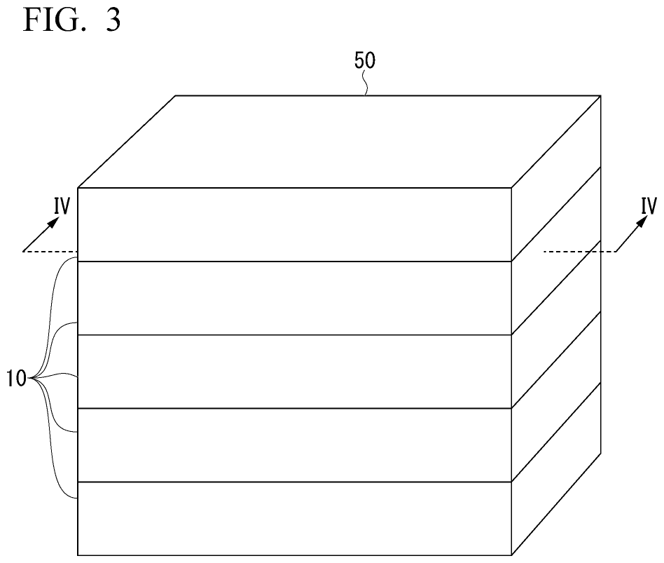

FIG. 2 is a cross-sectional view of the resin sheet and the resin substrate taken along line II-II shown in FIG. 1. FIG. 2 shows a cross section of the resin sheet 12 cut along the thickness direction. The resin sheet 12 contains a core material 30 and a resin component 22 which is impregnated into the core material 30 and covers both surfaces of the core material 30. ∘ in FIG. 2 indicates a glass fiber contained in the core material 30. The resin component 22 may be an uncured resin composition, or a part or the whole of the resin component 22 may be a semi-cured product of a resin composition.

Examples of the core material 30 include a woven fabric or a nonwoven fabric. Materials for the woven fabric and the nonwoven fabric are not limited to, for example, the glass fiber shown in FIG. 2, and examples thereof include at least one fiber selected from a glass fiber, a carbon fiber, a metal fiber, a natural fiber, and a synthetic fiber such as a polyester fiber or a polyamide fiber.

The resin sheet 12 can be manufactured as follows.

The core material 30 is impregnated with the resin composition by a technique such as coating or dipping. When the resin composition contains a solvent, the core material 30 is impregnated with the resin composition, and then heated and dried to remove the solvent. Heating conditions for removing the solvent in the resin composition may be, for example, 60° C. to 150° C. for about 1 to 120 minutes and are preferably 70° C. to 120° C. for about 3 to 90 minutes.

When a part or the whole of the resin component 22 contained in the resin sheet 12 is a semi-cured product of the resin composition, at the same time as heating for removing the solvent in the resin composition, a part or the whole of the resin composition impregnated into the core material 30 is cured to reach a semi-cured state. Moreover, after heating for removing the solvent in the resin composition, a part or the whole of the resin composition impregnated into the core material 30 may be cured to reach a semi-cured state under the same conditions as the heating for removing the solvent in the resin composition.

Through the aforementioned steps, the resin sheet 12, which has the resin component 22 composed of the uncured or at least partially semi-cured resin composition, is obtained.

Since the resin sheet 12 shown in FIG. 1 is obtained by molding the resin composition according to the present embodiment, a resin cured product having high thermal conductivity is obtained by heat-treating the resin sheet 12 to cure the resin composition. Accordingly, the resin sheet 12 shown in FIG. 1 is suitable as a material for a resin substrate.

The resin sheet 12 according to the present embodiment can be used as a precursor of a resin substrate (resin cured product) including a cured product of the resin composition.

In addition, in the present embodiment, as the resin sheet 12, the resin sheet having the core material 30 has been described as an example, as shown in FIG. 2, but the resin sheet according to the present invention may be formed only of a resin component without a core material.

Moreover, a metal foil such as a copper foil may be laminated on the surface of the resin sheet.

### “Resin Cured Product”

The resin substrate 10 (resin cured product) according to the present embodiment shown in FIGS. 1 and 2 is obtained by thermally curing the resin component 22 contained in the resin sheet 12, and includes a cured product 20 of the resin composition according to the present embodiment.

The resin substrate 10 according to the present embodiment can be manufactured by a method for using the aforementioned resin sheet 12 according to the present embodiment as a precursor and heating the resin sheet 12.

Specifically, the cured product 20 is obtained by heating the resin sheet 12 according to the present embodiment to thermally cure the uncured or semi-cured resin component 22. Heating conditions for curing the resin component 22 are preferably, for example, 100° C. to 250° C. for about 1 to 300 minutes. The heating for curing the resin component 22 may be performed under pressurization or reduced pressure, as needed.

The resin substrate 10 according to the present embodiment is a resin cured product that includes the cured product of the resin composition according to the present embodiment, and thus has high thermal conductivity.

In the present embodiment, as the resin substrate 10 (resin cured product), the resin substrate including the core material 30 and the cured product 20 covering the core material 30 has been described as an example, as shown in FIG. 2, but the resin cured product and the resin substrate according to the present invention may be formed only of a cured product of a resin composition.

Moreover, the resin cured product and the resin substrate according to the present invention may be manufactured by heating an amorphous resin composition, for example, as in the case of using the resin composition as an adhesive.

### “Laminated Substrate”

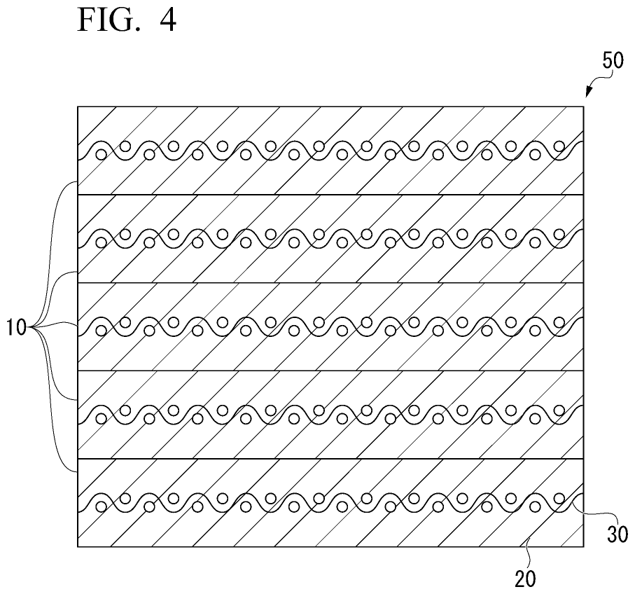

FIG. 3 is a perspective view showing an example of the laminated substrate according to the present invention. FIG. 4 is a cross-sectional view of the laminated substrate taken along line IV-IV shown in FIG. 3. FIG. 4 shows a cross section of the laminated substrate cut along the lamination direction. As shown in FIGS. 3 and 4, in a laminated substrate 50, a plurality of the resin substrates 10 shown in FIG. 2 are laminated and integrated.

The laminated substrate 50 can be manufactured, for example, by a method for heating the plurality of resin substrates 10 in an overlapped state. The laminated substrate 50 may be manufactured by a method for obtaining the cured product 20 by heating a plurality of resin sheets 12 in an overlapped state to thermally cure the uncured or semi-cured resin component. Heating conditions for the plurality of resin substrates 10 and heating conditions for the plurality of resin sheets 12 may be, fo4-16r example, 100° C. to 250° C. for about 1 to 300 minutes.

When heating the plurality of resin substrates 10 or the plurality of resin sheets 12, pressurization may be performed as needed. Pressurization conditions may be, for example, about 0.1 to 10 MPa. The pressurization is not essential when heating the plurality of resin substrates 10 or the plurality of resin sheets 12. Moreover, the heating of the plurality of resin substrates 10 or the plurality of resin sheets 12 may be performed under reduced pressure or vacuum.

The laminated substrate 50 according to the present embodiment is obtained by laminating the resin substrates 10, and thus has high thermal conductivity.

In the present embodiment, as the laminated substrate 50, the laminated substrate obtained by laminating the plurality of resin substrates 10, which are shown in FIG. 2 and include the cured product 20 of the resin composition, has been described as an example, but the laminated substrate according to the present invention may be any laminated substrate as long as at least one among the plurality of resin substrates is a resin substrate including a cured product of the resin composition according to the present invention.

Furthermore, the laminated substrate according to the present invention may be, for example, a metal-clad laminate having a metal layer on the upper surface and/or the lower surface. In this case, various known layers can be appropriately selected and used as the metal layer. Specifically, for example, a metal plate or metal foil made of a metal such as copper, nickel, or aluminum or the like can be used as the metal layer. The thickness of the metal layer is not particularly limited, and may be, for example, about 3 to 150 μm. A metal plate or metal foil subjected to etching and/or piercing may be used as the metal layer.

Hereinbefore, the embodiments of the present invention have been described in detail with reference to the drawings, but each configuration in each embodiment, a combination thereof, or the like is an example, and the additions, omissions, substitutions, and other modifications of the configuration can be made without departing from the spirit of the present invention.

### EXAMPLES

### Synthesis of Compound Represented by General Formula (1)

### Synthesis Examples 1 to 9

The first raw material and the second raw material shown in Table 1 were weighed into a three-necked flask in the ratio shown in Table 1, and dissolved in 1 L of tetrahydrofuran (THF) to obtain a first mixed solution. Thereafter, the first mixed solution was refluxed in a nitrogen stream to remove dissolved oxygen in the first mixed solution. Then, potassium carbonate was added to the first mixed solution in the ratio shown in Table 1, and a reaction was performed while maintaining the reflux state for 12 hours.

TABLE 1

| | | | | | | First raw | Second raw | Potassium |
|---|---|---|---|---|---|---|---|---|
| | | First raw | Second raw | Third raw | Fourth raw | material | material | carbonate |
| | | material | material | material | material | mol | mol | mol |
| Synthesis Example 1 | Formula (1) | 1-1 | 2-1 | 3-1 | 4-1 | 0.400 | 0.500 | 0.800 |
| Synthesis Example 2 | Formula (1) | 1-1 | 2-1 | 3-2 | 4-1 | 0.400 | 0.500 | 0.800 |
| Synthesis Example 3 | Formula (1) | 1-1 | 2-1 | 3-3 | 4-1 | 0.400 | 0.500 | 0.800 |
| Synthesis Example 4 | Formula (1) | 1-1 | 2-1 | 3-4 | 4-1 | 0.400 | 0.500 | 0.800 |
| Synthesis Example 5 | Formula (1) | 1-1 | 2-1 | 3-5 | 4-1 | 0.400 | 0.500 | 0.800 |
| Synthesis Example 6 | Formula (1) | 1-1 | 2-1 | 3-6 | 4-1 | 0.400 | 0.500 | 0.800 |
| Synthesis Example 7 | Formula (1) | 1-1 | 2-1 | 3-7 | 4-1 | 0.400 | 0.500 | 0.800 |
| Synthesis Example 8 | Formula (1) | 1-1 | 2-1 | 3-8 | 4-1 | 0.400 | 0.500 | 0.800 |
| Synthesis Example 9 | Formula (1) | 1-1 | 2-1 | 3-9 | 4-1 | 0.400 | 0.500 | 0.800 |
| Synthesis Example 10 | Formula (1) | 1-1 | 2-1 | 3-10 | 4-1 | 0.400 | 0.500 | 0.800 |
| Synthesis Example 11 | Formula (1) | 1-1 | 2-1 | 3-11 | 4-1 | 0.400 | 0.500 | 0.800 |
| Synthesis Example 12 | Formula (1) | 1-1 | 2-1 | 3-12 | 4-1 | 0.400 | 0.500 | 0.800 |
| Synthesis Example 13 | Formula (1) | 1-1 | 2-1 | 3-13 | 4-1 | 0.400 | 0.500 | 0.800 |
| Synthesis Example 14 | Formula (1) | 1-1 | 2-1 | 3-5 | 4-2 | 0.400 | 0.500 | 0.800 |
| Synthesis Example 15 | Formula (1) | 1-1 | 2-1 | 3-5 | 4-3 | 0.400 | 0.500 | 0.800 |
| Synthesis Example 16 | Formula (1) | 1-1 | 2-1 | 3-5 | 4-4 | 0.400 | 0.500 | 0.800 |
| Synthesis Example 17 | Formula (1) | 1-1 | 2-1 | 3-5 | 4-5 | 0.400 | 0.500 | 0.800 |
| Synthesis Example 18 | Formula (1) | 1-1 | 2-1 | 3-5 | 4-5 | 0.400 | 0.500 | 0.800 |
| Synthesis Example 19 | Formula (1) | 1-1 | 2-1 | 3-5 | 4-6 | 0.400 | 0.500 | 0.800 |

After the completion of the reaction, the obtained suspension was poured into water, and stirred for 30 minutes, and the produced precipitates were collected by filtration. The collected precipitates were vacuum-dried for 12 hours or longer, dissolved in chloroform, and subjected to silica gel chromatography to collect a first effluence. The solvent was removed from the first effluence to obtain a first precursor compound of each of Synthesis Examples 1 to 19. The obtained first precursor compound was subjected to size exclusion chromatography (SEC) analysis to determine the number-average molecular weight (Mn).

Subsequently, the first precursor compound (0.02 mol) and the third raw material (0.02 mol) shown in Table 1 were weighed into a three-necked flask, and dissolved in 1 L of tetrahydrofuran (THF) to obtain a second mixed solution. Thereafter, the second mixed solution was refluxed in a nitrogen stream to remove dissolved oxygen in the second mixed solution. Then, potassium carbonate (0.02 mol) was added to the second mixed solution, and a reaction was performed while maintaining the reflux state for 12 hours.

After the completion of the reaction, the obtained suspension was poured into water, and stirred for 30 minutes, and the produced precipitates were collected by filtration. The collected precipitates were vacuum-dried for 12 hours, dissolved in chloroform, and subjected to silica gel chromatography to collect a second effluence. The solvent was removed from the second effluence to obtain a second precursor compound of each of Synthesis Examples 1 to 19. The obtained second precursor compound was subjected to SEC analysis to determine the number-average molecular weight (Mn).

Subsequently, the second precursor compound (0.01 mol) and the fourth raw material (0.01 mol) shown in Table 1 were weighed into a three-necked flask, and dissolved in 1 L of tetrahydrofuran (THF) to obtain a third mixed solution. Thereafter, the third mixed solution was refluxed in a nitrogen stream to remove dissolved oxygen in the third mixed solution. Then, potassium carbonate (0.01 mol) was added to the third mixed solution, and a reaction was performed while maintaining the reflux state for 12 hours.

After the completion of the reaction, the obtained suspension was poured into water, and stirred for 30 minutes, and the produced precipitates were collected by filtration. The collected precipitates were vacuum-dried for 12 hours to obtain compounds of Synthesis Examples 1 to 17 and 19 represented by General Formula (1).

50 g of the compound of Synthesis Example 17 obtained as described above was added to 500 mL of N-methyl-2-pyrrolidone (NMP), and heated to 100° C. to dissolve the compound of Synthesis Example 17, the heating was then stopped, and 10 mL of concentrated aqueous ammonia (15 mol/L) was added dropwise. The obtained mixed liquid was stirred for 1 hour and the solvent was distilled off under reduced pressure. The obtained residue was vacuum-dried at 120° C. for 12 hours to obtain a compound of Synthesis Example 18 represented by General Formula (1), which is a target substance.

### Synthesis of Compound Represented by General Formula (2)

### Synthesis Examples 20 to 39

A first precursor compound of each of Synthesis Examples 20 to 39 was obtained in the same manner as the first precursor compound of Synthesis Example 1, except that the first raw material, the second raw material, and the potassium carbonate shown in Table 2 were used in the ratio shown in Table 2. The obtained first precursor compound was subjected to size exclusion chromatography (SEC) analysis to determine the number-average molecular weight (Mn).

TABLE 2

| | | | | | | First raw | Second raw | Potassium |
|---|---|---|---|---|---|---|---|---|
| | | First raw | Second raw | Third raw | Fourth raw | material | material | carbonate |
| | | material | material | material | material | mol | mol | mol |
| Synthesis Example 20 | Formula (2) | 1-1 | 2-1 | 3-14 | 4-7 | 0.500 | 0.400 | 0.800 |
| Synthesis Example 21 | Formula (2) | 1-1 | 2-1 | 3-14 | 4-7 | 0.500 | 0.400 | 0.800 |
| Synthesis Example 22 | Formula (2) | 1-1 | 2-1 | 3-14 | 4-8 | 0.500 | 0.400 | 0.800 |
| Synthesis Example 23 | Formula (2) | 1-1 | 2-1 | 3-14 | 4-9 | 0.500 | 0.400 | 0.800 |
| Synthesis Example 24 | Formula (2) | 1-1 | 2-1 | 3-14 | 4-10 | 0.500 | 0.400 | 0.800 |
| Synthesis Example 25 | Formula (2) | 1-1 | 2-1 | 3-14 | 4-11 | 0.500 | 0.400 | 0.800 |
| Synthesis Example 26 | Formula (2) | 1-1 | 2-1 | 3-14 | 4-12 | 0.500 | 0.400 | 0.800 |
| Synthesis Example 27 | Formula (2) | 1-1 | 2-1 | 3-14 | 4-13 | 0.500 | 0.400 | 0.800 |
| Synthesis Example 28 | Formula (2) | 1-1 | 2-1 | 3-14 | 4-14 | 0.500 | 0.400 | 0.800 |
| Synthesis Example 29 | Formula (2) | 1-1 | 2-1 | 3-14 | 4-15 | 0.500 | 0.400 | 0.800 |
| Synthesis Example 30 | Formula (2) | 1-1 | 2-1 | 3-14 | 4-16 | 0.500 | 0.400 | 0.800 |
| Synthesis Example 31 | Formula (2) | 1-1 | 2-1 | 3-14 | 4-17 | 0.500 | 0.400 | 0.800 |
| Synthesis Example 32 | Formula (2) | 1-1 | 2-1 | 3-14 | 4-18 | 0.500 | 0.400 | 0.800 |
| Synthesis Example 33 | Formula (2) | 1-1 | 2-1 | 3-14 | 4-19 | 0.500 | 0.400 | 0.800 |
| Synthesis Example 34 | Formula (2) | 1-1 | 2-1 | 3-14 | 4-20 | 0.500 | 0.400 | 0.800 |
| Synthesis Example 35 | Formula (2) | 1-1 | 2-1 | 3-14 | 4-21 | 0.500 | 0.400 | 0.800 |
| Synthesis Example 36 | Formula (2) | 1-1 | 2-1 | 3-14 | 4-21 | 0.500 | 0.400 | 0.800 |
| Synthesis Example 37 | Formula (2) | 1-1 | 2-1 | 3-14 | 4-22 | 0.500 | 0.400 | 0.800 |
| Synthesis Example 38 | Formula (2) | 1-1 | 2-1 | 3-14 | 4-23 | 0.500 | 0.400 | 0.800 |
| Synthesis Example 39 | Formula (2) | 1-1 | 2-1 | 3-14 | 4-24 | 0.500 | 0.400 | 0.800 |

Subsequently, the first precursor compound (0.02 mol) and the fourth raw material (0.02 mol) shown in Table 2 were weighed into a three-necked flask, and dissolved in 1 L of tetrahydrofuran (THF) to obtain a second mixed solution. Thereafter, the second mixed solution was refluxed in a nitrogen stream to remove dissolved oxygen in the second mixed solution. Then, potassium carbonate (0.02 mol) was added to the second mixed solution, and a reaction was performed while maintaining the reflux state for 12 hours.

After the completion of the reaction, the obtained suspension was poured into water, and stirred for 30 minutes, and the produced precipitates were collected by filtration. The collected precipitates were vacuum-dried for 12 hours, dissolved in chloroform, and subjected to silica gel chromatography to collect a second effluence. The solvent was removed from the second effluence to obtain a second precursor compound of each of Synthesis Examples 20 to 39. The obtained second precursor compound was subjected to SEC analysis to determine the number-average molecular weight (Mn).

Subsequently, the second precursor compound (0.01 mol) and the third raw material (0.01 mol) shown in Table 2 were weighed into a three-necked flask, and dissolved in 1 L of tetrahydrofuran (THF) to obtain a third mixed solution. Thereafter, the third mixed solution was refluxed in a nitrogen stream to remove dissolved oxygen in the third mixed solution. Then, potassium carbonate (0.01 mol) was added to the third mixed solution, and a reaction was performed while maintaining the reflux state for 12 hours.

After the completion of the reaction, the obtained suspension was poured into water, and stirred for 30 minutes, and the produced precipitates were collected by filtration. The collected precipitates were vacuum-dried for 12 hours to obtain a third precursor compound of each of Synthesis Examples 20 to 39. The obtained third precursor compound was subjected to SEC analysis to determine the number-average molecular weight (Mn).

Subsequently, the third precursor compound (0.01 mol) of each of Synthesis Examples 20 to 36 was weighed into a three-necked flask, and dissolved in 1 L of tetrahydrofuran (THF) to obtain a fourth mixed solution. Thereafter, the fourth mixed solution was refluxed in a nitrogen stream to remove dissolved oxygen in the fourth mixed solution. Then, potassium hydroxide (0.01 mol) and water (10 mL) were added to the fourth mixed solution, and a reaction was performed while maintaining the reflux state for 12 hours.

After the completion of the reaction, the obtained suspension was poured into water, neutralized with hydrochloric acid so as to have a pH of 2 or less, and stirred for 30 minutes, and the produced precipitates were collected by filtration. The collected precipitates were vacuum-dried for 12 hours to obtain compounds of Synthesis Examples and 22 to 36 represented by General Formula (2).

50 g of the compound of Synthesis Example 20 obtained as described above was added to 500 mL of N-methyl-2-pyrrolidone (NMP), and heated to 100° C. to dissolve the compound of Synthesis Example 20, the heating was then stopped, and 10 mL of concentrated aqueous ammonia (15 mol/L) was added dropwise. The obtained mixed liquid was stirred for 1 hour and the solvent was distilled off under reduced pressure. The obtained residue was vacuum-dried at 120° C. for 12 hours to obtain a compound of Synthesis Example 21 represented by General Formula (2), which is a target substance.

Moreover, the third precursor compound (0.01 mol) of each of Synthesis Examples 37 to 39 was weighed into a three-necked flask, and 1 L of benzyl alcohol and iron (3 g) were added to obtain a reaction mixture. The reaction mixture was heated to 80° C., concentrated hydrochloric acid (15 mL) was added over 30 minutes using a dropping funnel, the resultant was refluxed for 1 hour, and then the temperature of the resultant was returned to room temperature. The reaction mixture was poured into water, and 2 mol/L of a sodium hydroxide aqueous solution was added until the pH reached 7 or greater while stirring. Subsequently, extraction separation was performed using chloroform, the resultant was dried over sodium sulfate, and the precipitates collected by filtration were then distilled off under reduced pressure to obtain compounds of Synthesis Examples 37 to 39 represented by General Formula (2).

### Synthesis of Compound Represented by General Formula (3)

### Synthesis Examples 40 to 68

A first precursor compound of each of Synthesis Examples 40 to 68 was obtained in the same manner as the first precursor compound of Synthesis Example 1, except that the first raw material, the second raw material, and the potassium carbonate shown in Table 3 were used in the ratio shown in Table 3. The obtained first precursor compound was subjected to size exclusion chromatography (SEC) analysis to determine the number-average molecular weight (Mn).

TABLE 3

| | | | | | | First raw | Second raw | Potassium |
|---|---|---|---|---|---|---|---|---|
| | | First raw | Second raw | Third raw | Fourth raw | material | material | carbonate |
| | | material | material | material | material | mol | mol | mol |
| Synthesis Example 40 | Formula (3) | 1-1 | 2-1 | None | None | 0.500 | 0.500 | 1.05 |
| Synthesis Example 41 | Formula (3) | 1-2 | 2-1 | None | None | 0.500 | 0.500 | 1.05 |
| Synthesis Example 42 | Formula (3) | 1-2 | 2-2 | None | None | 0.500 | 0.500 | 1.05 |
| Synthesis Example 43 | Formula (3) | 1-1 | 2-2 | None | None | 0.500 | 0.500 | 1.05 |
| Synthesis Example 44 | Formula (3) | 1-3 | 2-1 | None | None | 0.500 | 0.500 | 1.05 |
| Synthesis Example 45 | Formula (3) | 1-2 | 2-3 | None | None | 0.500 | 0.500 | 1.05 |
| Synthesis Example 46 | Formula (3) | 1-3 | 2-3 | None | None | 0.500 | 0.500 | 1.05 |
| Synthesis Example 47 | Formula (3) | 1-4 | 2-1 | None | None | 0.500 | 0.500 | 1.05 |
| Synthesis Example 48 | Formula (3) | 1-5 | 2-1 | None | None | 0.500 | 0.500 | 1.05 |
| Synthesis Example 49 | Formula (3) | 1-6 | 2-1 | None | None | 0.500 | 0.500 | 1.05 |
| Synthesis Example 50 | Formula (3) | 1-7 | 2-1 | None | None | 0.500 | 0.500 | 1.05 |
| Synthesis Example 51 | Formula (3) | 1-8 | 2-1 | None | None | 0.500 | 0.500 | 1.05 |
| Synthesis Example 52 | Formula (3) | 1-9 | 2-1 | None | None | 0.500 | 0.500 | 1.05 |
| Synthesis Example 53 | Formula (3) | 1-1 | 2-4 | None | None | 0.500 | 0.500 | 1.05 |
| Synthesis Example 54 | Formula (3) | 1-1 | 2-5 | None | None | 0.500 | 0.500 | 1.05 |
| Synthesis Example 55 | Formula (3) | 1-1 | 2-6 | None | None | 0.500 | 0.500 | 1.05 |
| Synthesis Example 56 | Formula (3) | 1-1 | 2-7 | None | None | 0.500 | 0.500 | 1.05 |
| Synthesis Example 57 | Formula (3) | 1-10 | 2-1 | None | None | 0.500 | 0.500 | 1.05 |
| Synthesis Example 58 | Formula (3) | 1-11 | 2-1 | None | None | 0.500 | 0.500 | 1.05 |
| Synthesis Example 59 | Formula (3) | 1-12 | 2-1 | None | None | 0.500 | 0.500 | 1.05 |
| Synthesis Example 60 | Formula (3) | 1-13 | 2-1 | None | None | 0.500 | 0.500 | 1.05 |
| Synthesis Example 61 | Formula (3) | 1-1 | 2-8 | None | None | 0.500 | 0.500 | 1.05 |
| Synthesis Example 62 | Formula (3) | 1-1 | 2-9 | None | None | 0.500 | 0.500 | 1.05 |
| Synthesis Example 63 | Formula (3) | 1-1 | 2-10 | None | None | 0.500 | 0.500 | 1.05 |
| Synthesis Example 64 | Formula (3) | 1-11 | 2-10 | None | None | 0.500 | 0.500 | 1.05 |
| Synthesis Example 65 | Formula (3) | 1-1 | 2-1 | None | None | 0.500 | 0.500 | 1.04 |
| Synthesis Example 66 | Formula (3) | 1-1 | 2-1 | None | None | 0.500 | 0.500 | 1.03 |
| Synthesis Example 67 | Formula (3) | 1-1 | 2-1 | None | None | 0.500 | 0.500 | 1.02 |
| Synthesis Example 68 | Formula (3) | 1-1 | 2-11 | None | None | 0.500 | 0.500 | 1.05 |

Subsequently, the first precursor compound (0.01 mol) was weighed into a three-necked flask, and dissolved in 1 L of tetrahydrofuran (THF) to obtain a second mixed solution. Then, sodium hydrogen carbonate (0.01 mol) and 10 mL of water were added to the second mixed solution, and a reaction was performed while maintaining the reflux state for 12 hours.

After the completion of the reaction, the obtained suspension was poured into water, and stirred for 30 minutes, and the produced precipitates were collected by filtration. The collected precipitates were vacuum-dried for 12 hours to obtain compounds of Synthesis Examples 40 to 68 represented by General Formula (3).

### Synthesis of Compound Represented by General Formula (4)

### Synthesis Examples 69 to 75

A first precursor compound of each of Synthesis Examples 69 to 75 was obtained in the same manner as the first precursor compound of Synthesis Example 1, except that the first raw material, the second raw material, and the potassium carbonate shown in Table 4 were used in the ratio shown in Table 4. The obtained first precursor compound was subjected to size exclusion chromatography (SEC) analysis to determine the number-average molecular weight (Mn).

TABLE 4

| | | | | | | First raw | Second raw | Potassium |
|---|---|---|---|---|---|---|---|---|
| | | First raw | Second raw | Third raw | Fourth raw | material | material | carbonate |
| | | material | material | material | material | mol | mo | mol |
| Synthesis Example 69 | Formula (4) | 1-1 | 2-1 | None | 4-1 | 0.400 | 0.500 | 0.800 |
| Synthesis Example 70 | Formula (4) | 1-1 | 2-1 | None | 4-2 | 0.400 | 0.500 | 0.800 |
| Synthesis Example 71 | Formula (4) | 1-1 | 2-1 | None | 4-3 | 0.400 | 0.500 | 0.800 |
| Synthesis Example 72 | Formula (4) | 1-1 | 2-1 | None | 4-4 | 0.400 | 0.500 | 0.800 |
| Synthesis Example 73 | Formula (4) | 1-1 | 2-1 | None | 4-5 | 0.400 | 0.500 | 0.800 |
| Synthesis Example 74 | Formula (4) | 1-1 | 2-1 | None | 4-5 | 0.400 | 0.500 | 0.800 |
| Synthesis Example 75 | Formula (4) | 1-1 | 2-1 | None | 4-6 | 0.400 | 0.500 | 0.800 |

Subsequently, the first precursor compound (0.02 mol) and the fourth raw material (0.02 mol) shown in Table 4 were weighed into a three-necked flask, and dissolved in 1 L of tetrahydrofuran (THF) to obtain a second mixed solution. Thereafter, the second mixed solution was refluxed in a nitrogen stream to remove dissolved oxygen in the second mixed solution. Then, potassium carbonate (0.02 mol) was added to the second mixed solution, and a reaction was performed while maintaining the reflux state for 12 hours.

After the completion of the reaction, the obtained suspension was poured into water, and stirred for 30 minutes, and the produced precipitates were collected by filtration. The collected precipitates were vacuum-dried for 12 hours, dissolved in chloroform, and subjected to silica gel chromatography to collect a second effluence. The solvent was removed from the second effluence to obtain a second precursor compound of each of Synthesis Examples 69 to 75. The obtained second precursor compound was subjected to SEC analysis to determine the number-average molecular weight (Mn).

Subsequently, the second precursor compound was weighed into a three-necked flask, and dissolved in 1 L of tetrahydrofuran (THF) to obtain a third mixed solution. Thereafter, the third mixed solution was refluxed in a nitrogen stream to remove dissolved oxygen in the third mixed solution. Then, sodium hydrogen carbonate (0.01 mol) and 10 mL of water were added to the third mixed solution, and a reaction was performed while maintaining the reflux state for 12 hours.

After the completion of the reaction, the obtained suspension was poured into water, and stirred for 30 minutes, and the produced precipitates were collected by filtration. The collected precipitates were vacuum-dried for 12 hours to obtain compounds of Synthesis Examples 69 to 73 and 75 represented by General Formula (4).

50 g of the compound of Synthesis Example 73 obtained as described above was added to 500 mL of N-methyl-2-pyrrolidone (NMP), and heated to 100° C. to dissolve the compound of Synthesis Example 73, the heating was then stopped, and 10 mL of concentrated aqueous ammonia (15 mol/L) was added dropwise. The obtained mixed liquid was stirred for 1 hour and the solvent was distilled off under reduced pressure. The obtained residue was vacuum-dried at 120° C. for 12 hours to obtain a compound of Synthesis Example 74 represented by General Formula (4), which is a target substance.

1-1 to 1-13 in the first raw materials shown in Tables 1 to 4 are the following compounds.

### [First Raw Material]

(1-1) Methylhydroquinone
(1-2) Hydroquinone
(1-3) Tetramethylhydroquinone
(1-4) Trimethylhydroquinone
(1-5) 2-(Trifluoromethyl)-1,4-benzenediol
(1-6) Fluorohydroquinone
(1-7) Chlorohydroquinone
(1-8) Bromohydroquinone
(1-9) 2,5-Dihydroxynitrobenzene
(1-10) 1,5-Dihydroxynaphthalene
(1-11) 2,6-Dihydroxynaphthalene
(1-12) 3,3′,5,5′-Tetramethylbiphenyl-4,4′-diol
(1-13) 4,4′-Dihydroxybiphenyl

2-1 to 2-11 in the second raw materials shown in Tables 1 to 4 are the following compounds.

### [Second Raw Material]

(2-1) α,α′-Dichloro-p-xylene (α,α′-p-Dichloroxylene)
(2-2) 1,4-Bis(chloromethyl)-2-methylbenzene
(2-3) 3,6-Bis(chloromethyl)durene
(2-4) 1,4-Bis(bromomethyl)-2-fluorobenzene
(2-5) 1,4-Bis(bromomethyl)-2-chlorobenzene
(2-6) 2-Bromo-1,4-bis(bromomethyl)benzene
(2-7) 1,4-Bis(chloromethyl)-2-nitrobenzene
(2-8) 4,4′-Bis(chloromethyl)biphenyl
(2-9) 2,6-Bis(bromomethyl)naphthalene
(2-10) 1,5-Bis(chloromethyl)naphthalene
(2-11) 1,4-Bis(bromomethyl)naphthalene

3-1 to 3-14 in the third raw materials shown in Tables 1 and 2 are the following compounds.

### [Third Raw Material]

(3-1) 2,6-Difluoro-4-hydroxy-benzyl alcohol
(2,6-Difluoro-4-hydroxy-benzenemethanol)
(3-2) 2-Fluoro-4-hydroxy-benzyl alcohol
(2-Fluoro-4-hydroxy-benzenemethanol)
(3-3) 3-Bromo-4-hydroxy-benzyl alcohol
(3-Bromo-4-hydroxy-benzenemethanol)
(3-4) 4-Hydroxy-3-nitro-benzyl alcohol (4-Hydroxy-3-nitro-benzenemethanol)
(3-5) 4-Hydroxy-3-methyl-benzyl alcohol
(4-Hydroxy-3-methyl-benzenemethanol)
(3-6) 4-Hydroxy-3,5-dimethyl-benzyl alcohol
(4-Hydroxy-3,5-dimethyl-benzenemethanol)
(3-7) 2-Chloro-4-hydroxy-benzyl alcohol
(2-Chloro-4-hydroxy-benzenemethanol)
(3-8) 3,5-Difluoro-4-hydroxy-benzyl alcohol
(3,5-Difluoro-4-hydroxy-benzenemethanol)
(3-9) 4-Hydroxy-2,6-dimethyl-benzyl alcohol
(4-Hydroxy-2,6-dimethyl-benzenemethanol)
(3-10) 4-Hydroxy-2-methyl-benzyl alcohol
(4-Hydroxy-2-methyl-benzenemethanol)
(3-11) 4-Hydroxy-2-nitro-benzyl alcohol (4-Hydroxy-2-nitro-benzenemethanol)
(3-12) 4-Hydroxy-3-(trifluoromethyl)-benzyl alcohol
(4-Hydroxy-3-(trifluoromethyl)-benzenemethanol)
(3-13) 4-Hydroxy-2,5-dimethyl-benzyl alcohol
(4-Hydroxy-2,5-dimethyl-benzenemethanol)
(3-14) 5-(Bromomethyl)-2-hydroxymethyl-2-benzonitrile
(5-(Bromomethyl)-2-(hydroxymethyl)-benzonitrile)

4-1 to 4-24 in the fourth raw materials shown in Tables 1, 2, and 4 are the following compounds.

### [Fourth Raw Material]

(4-1) Hydroquinone
(4-2) 2-Fluoro-1,4-benzenediol
(4-3) 2,3,5,6-Tetrafluoro-1,4-benzenediol
(4-4) 2,3-Difluoro-1,4-benzenediol
(4-5) Methyl 4-hydroxybenzoate
(4-6) p-Aminophenol (4-Aminophenol)
(4-7) 4-(Bromomethyl)-benzoic acid methyl ester
(4-8) 4-(Bromomethyl)-3-fluoro-benzoic acid methyl ester
(4-9) 2-Bromo-4-(bromomethyl)-benzoic acid methyl ester
(4-10) 4-(Bromomethyl)-3-chloro-benzoic acid methyl ester
(4-11) 4-(Chloromethyl)-3,5-difluoro-benzoic acid methyl ester
(4-12) 4-(Bromomethyl)-2-methyl-benzoic acid methyl ester
(4-13) 4-(Bromomethyl)-3-nitro-benzoic acid methyl ester
(4-14) 2-Chloro-4-(chloromethyl)-6-cyano-benzoic acid ethyl ester
(4-15) 4-(Bromomethyl)-2,6-difluoro-benzoic acid methyl ester
(4-16) 4-(Bromomethyl)-3-(trifluoromethyl)-benzoic acid methyl ester
(4-17) 4-(Bromomethyl)-2,5-difluoro-benzoic acid methyl ester
(4-18) 4-(Bromomethyl)-3-cyano-benzoic acid methyl ester
(4-19) 5-Bromo-4-(bromomethyl)-2-fluoro-benzoic acid methyl ester
(4-20) 4-(Bromomethyl)-2-nitro-benzoic acid methyl ester
(4-21) 4-(Bromomethyl)-2,3-difluoro-benzoic acid methyl ester
(4-22) Benzene, 1-(bromomethyl)-4-nitro-
(4-23) Benzene, 1-(bromomethyl)-4-nitro-2-(trifluoromethyl)-
(4-24) Benzene, 4-(bromomethyl)-2-methyl-1-nitro-

The compounds of Synthesis Examples 1 to 75 obtained as described above were analyzed by size exclusion chromatography (SEC method) using a molecular weight analyzer (GPC-104, manufactured by Shodex).

A sample solution for analysis was prepared by the following method. 2 mg of each of the compounds of Synthesis Examples 1 to 75 was taken, added to 10 mL of tetrahydrofuran (THF), and shaken with a vibrator for 7 hours. The shaken solution was heated in an oven at 55° C. for 2 hours and allowed to stand still. Thereafter, the solution was filtered using a polytetrafluoroethylene (PTFE) filter having a pore size of 0.45 μm, and the filtrate was used as a sample solution for analysis.

The analysis was performed by connecting four columns (manufactured by Shodex, columns for SEC (GPC): KF-403HQ, KF-402.5HQ, KF-402HQ, and KF-401HQ) and maintaining the column temperature at 40° C. THF (containing 0.03% by mass of dibutylhydroxytoluene (BHT)) was used as the mobile phase and flowed at a flow rate of 0.3 ml/min. A detector for ultraviolet rays (UV) having a wavelength of 254 nm was used as a detector. Polystyrene was used as a standard substance.

From the results of such analysis, the number-average molecular weight (Mn), weight-average molecular weight (Mw), Mw/Mn, minimum molecular weight (Mn), and maximum molecular weight (Mn) of each of the compounds of Synthesis Examples 1 to 75 were determined. The results thereof are shown in Tables 5 to 8.

TABLE 5

| | | | | | Minimum | Maximum |
|---|---|---|---|---|---|---|
| | | | | | molecular | molecular |
| | | Mn | Mw | Mw/Mn | weight | weight |
| Synthesis | Formula | 1587 | 3496 | 2.20 | 479 | 37491 |
| Example 1 | (1) | | | | | |
| Synthesis | Formula | 1634 | 4355 | 2.67 | 546 | 47536 |
| Example 2 | (1) | | | | | |
| Synthesis | Formula | 1680 | 5289 | 3.15 | 510 | 36464 |
| Example 3 | (1) | | | | | |
| Synthesis | Formula | 1562 | 3622 | 2.32 | 488 | 33490 |
| Example 4 | (1) | | | | | |
| Synthesis | Formula | 1695 | 4416 | 2.61 | 601 | 36264 |
| Example 5 | (1) | | | | | |
| Synthesis | Formula | 1422 | 2570 | 1.81 | 512 | 30051 |
| Example 6 | (1) | | | | | |
| Synthesis | Formula | 1472 | 4659 | 3.17 | 566 | 30186 |
| Example 7 | (1) | | | | | |
| Synthesis | Formula | 1628 | 4680 | 2.87 | 580 | 33825 |
| Example 8 | (1) | | | | | |
| Synthesis | Formula | 1422 | 3775 | 2.65 | 407 | 36234 |
| Example 9 | (1) | | | | | |
| Synthesis | Formula | 1495 | 3776 | 2.53 | 519 | 30446 |
| Example 10 | (1) | | | | | |
| Synthesis | Formula | 1588 | 3106 | 1.96 | 459 | 31936 |
| Example 11 | (1) | | | | | |
| Synthesis | Formula | 1605 | 4156 | 2.59 | 500 | 40021 |
| Example 12 | (1) | | | | | |
| Synthesis | Formula | 1452 | 2811 | 1.94 | 456 | 45708 |
| Example 13 | (1) | | | | | |
| Synthesis | Formula | 1475 | 3455 | 2.34 | 446 | 40258 |
| Example 14 | (1) | | | | | |
| Synthesis | Formula | 1675 | 3367 | 2.01 | 519 | 48615 |
| Example 15 | (1) | | | | | |
| Synthesis | Formula | 1622 | 4210 | 2.60 | 500 | 40073 |
| Example 16 | (1) | | | | | |
| Synthesis | Formula | 1664 | 4420 | 2.66 | 584 | 32870 |
| Example 17 | (1) | | | | | |
| Synthesis | Formula | 1680 | 4420 | 2.66 | 600 | 32880 |
| Example 18 | (1) | | | | | |
| Synthesis | Formula | 1565 | 4626 | 2.96 | 455 | 49608 |
| Example 19 | (1) | | | | | |

TABLE 6

| | | | | | Minimum | Maximum |
|---|---|---|---|---|---|---|
| | | | | | molecular | molecular |
| | | Mn | Mw | Mw/Mn | weight | weight |
| Synthesis | Formula | 1567 | 2713 | 1.73 | 482 | 47846 |
| Example 20 | (2) | | | | | |
| Synthesis | Formula | 1583 | 2730 | 1.73 | 496 | 47852 |
| Example 21 | (2) | | | | | |
| Synthesis | Formula | 1570 | 2794 | 1.78 | 487 | 33149 |
| Example 22 | (2) | | | | | |
| Synthesis | Formula | 1510 | 3014 | 2.00 | 598 | 47529 |
| Example 23 | (2) | | | | | |
| Synthesis | Formula | 1572 | 3687 | 2.35 | 453 | 44680 |
| Example 24 | (2) | | | | | |
| Synthesis | Formula | 1655 | 3973 | 2.40 | 528 | 45863 |
| Example 25 | (2) | | | | | |
| Synthesis | Formula | 1616 | 4606 | 2.85 | 465 | 44273 |
| Example 26 | (2) | | | | | |
| Synthesis | Formula | 1540 | 3098 | 2.01 | 446 | 30434 |
| Example 27 | (2) | | | | | |
| Synthesis | Formula | 1491 | 3326 | 2.23 | 545 | 43490 |
| Example 28 | (2) | | | | | |
| Synthesis | Formula | 1630 | 4054 | 2.49 | 649 | 48097 |
| Example 29 | (2) | | | | | |
| Synthesis | Formula | 1609 | 4782 | 2.97 | 604 | 43318 |
| Example 30 | (2) | | | | | |
| Synthesis | Formula | 1697 | 4632 | 2.73 | 527 | 35821 |
| Example 31 | (2) | | | | | |
| Synthesis | Formula | 1607 | 4051 | 2.52 | 523 | 32421 |
| Example 32 | (2) | | | | | |
| Synthesis | Formula | 1606 | 4088 | 2.55 | 628 | 44714 |
| Example 33 | (2) | | | | | |
| Synthesis | Formula | 1580 | 5049 | 3.20 | 484 | 42726 |
| Example 34 | (2) | | | | | |
| Synthesis | Formula | 1403 | 4776 | 3.40 | 450 | 46271 |
| Example 35 | (2) | | | | | |
| Synthesis | Formula | 1636 | 4675 | 2.86 | 471 | 49468 |
| Example 36 | (2) | | | | | |
| Synthesis | Formula | 1536 | 4432 | 2.89 | 451 | 31584 |
| Example 37 | (2) | | | | | |
| Synthesis | Formula | 1553 | 3388 | 2.18 | 488 | 31102 |
| Example 38 | (2) | | | | | |
| Synthesis | Formula | 1597 | 4306 | 2.70 | 463 | 35630 |
| Example 39 | (2) | | | | | |

TABLE 7

| | | | | | Minimum | Maximum |
|---|---|---|---|---|---|---|
| | | | | | molecular | molecular |
| | | Mn | Mw | Mw/Mn | weight | weight |
| Synthesis | Formula | 1432 | 3906 | 2.73 | 471 | 34238 |
| Example 40 | (3) | | | | | |
| Synthesis | Formula | 1156 | 2748 | 2.38 | 342 | 30742 |
| Example 41 | (3) | | | | | |
| Synthesis | Formula | 4167 | 11716 | 2.81 | 927 | 30406 |
| Example 42 | (3) | | | | | |
| Synthesis | Formula | 4408 | 8464 | 1.92 | 1145 | 40421 |
| Example 43 | (3) | | | | | |
| Synthesis | Formula | 3929 | 8897 | 2.26 | 888 | 49792 |
| Example 44 | (3) | | | | | |
| Synthesis | Formula | 4763 | 13331 | 2.80 | 1145 | 36471 |
| Example 45 | (3) | | | | | |
| Synthesis | Formula | 4372 | 10744 | 2.46 | 1040 | 37477 |
| Example 46 | (3) | | | | | |
| Synthesis | Formula | 4323 | 8281 | 1.92 | 994 | 30700 |
| Example 47 | (3) | | | | | |
| Synthesis | Formula | 3636 | 8041 | 2.21 | 847 | 31836 |
| Example 48 | (3) | | | | | |
| Synthesis | Formula | 3524 | 9595 | 2.72 | 838 | 37903 |
| Example 49 | (3) | | | | | |
| Synthesis | Formula | 4525 | 11397 | 2.52 | 1144 | 33944 |
| Example 50 | (3) | | | | | |
| Synthesis | Formula | 3237 | 7799 | 2.41 | 816 | 41098 |
| Example 51 | (3) | | | | | |
| Synthesis | Formula | 4738 | 11177 | 2.36 | 1089 | 30851 |
| Example 52 | (3) | | | | | |
| Synthesis | Formula | 4483 | 10262 | 2.29 | 1051 | 38258 |
| Example 53 | (3) | | | | | |
| Synthesis | Formula | 3582 | 7349 | 2.05 | 970 | 48752 |
| Example 54 | (3) | | | | | |
| Synthesis | Formula | 4155 | 10895 | 2.62 | 1118 | 45268 |
| Example 55 | (3) | | | | | |
| Synthesis | Formula | 4009 | 8867 | 2.21 | 988 | 33165 |
| Example 56 | (3) | | | | | |
| Synthesis | Formula | 4381 | 12004 | 2.74 | 1121 | 33487 |
| Example 57 | (3) | | | | | |
| Synthesis | Formula | 3481 | 7812 | 2.24 | 868 | 42884 |
| Example 58 | (3) | | | | | |
| Synthesis | Formula | 3599 | 8583 | 2.38 | 920 | 40847 |
| Example 59 | (3) | | | | | |
| Synthesis | Formula | 4353 | 8445 | 1.94 | 1171 | 42658 |
| Example 60 | (3) | | | | | |
| Synthesis | Formula | 3271 | 7001 | 2.14 | 779 | 32517 |
| Example 61 | (3) | | | | | |
| Synthesis | Formula | 4081 | 7984 | 1.96 | 1095 | 45609 |
| Example 62 | (3) | | | | | |
| Synthesis | Formula | 4898 | 16170 | 3.30 | 1248 | 35308 |
| Example 63 | (3) | | | | | |
| Synthesis | Formula | 4894 | 9562 | 1.95 | 1378 | 30164 |
| Example 64 | (3) | | | | | |
| Synthesis | Formula | 1771 | 3802 | 2.15 | 471 | 41909 |
| Example 65 | (3) | | | | | |
| Synthesis | Formula | 2350 | 7495 | 3.19 | 470 | 32889 |
| Example 66 | (3) | | | | | |
| Synthesis | Formula | 2844 | 9306 | 3.27 | 471 | 46109 |
| Example 67 | (3) | | | | | |
| Synthesis | Formula | 1692 | 4166 | 2.46 | 731 | 34498 |
| Example 68 | (3) | | | | | |

TABLE 8

| | | | | | Minimum | Maximum |
|---|---|---|---|---|---|---|
| | | | | | molecular | molecular |
| | | Mn | Mw | Mw/Mn | weight | weight |
| Synthesis | Formula | 2033 | 4344 | 2.14 | 682 | 34160 |
| Example 69 | (4) | | | | | |
| Synthesis | Formula | 1896 | 5364 | 2.83 | 632 | 36996 |
| Example 70 | (4) | | | | | |
| Synthesis | Formula | 2012 | 4621 | 2.30 | 783 | 34388 |
| Example 71 | (4) | | | | | |
| Synthesis | Formula | 1637 | 4302 | 2.63 | 639 | 37842 |
| Example 72 | (4) | | | | | |
| Synthesis | Formula | 1895 | 5101 | 2.69 | 559 | 30610 |
| Example 73 | (4) | | | | | |
| Synthesis | Formula | 1900 | 5120 | 2.69 | 575 | 30620 |
| Example 74 | (4) | | | | | |
| Synthesis | Formula | 1618 | 4180 | 2.58 | 560 | 36092 |
| Example 75 | (4) | | | | | |

Moreover, the structures of Synthesis Examples 1 to 75 identified from the above analysis results are shown below.

The compounds of Synthesis Examples 1 to 19 were compounds represented by General Formula (1). Ar₁, Ar₂, Ar₃, Ar₄, and Z in Formula (1) are shown in Table 9.

The compounds of Synthesis Examples 20 to 39 were compounds represented by General Formula (2). Ar₁, Ar₂, Ar₃, Ar₄, and Z in Formula (2) are shown in Table 10.

The compounds of Synthesis Examples 40 to 68 were compounds represented by General Formula (3). Ar₁, Ar₂, Ar₃, Ar₄, and Z in Formula (3) are shown in Table 11.

The compounds of Synthesis Examples 69 to 75 were compounds represented by General Formula (4). Ar₁, Ar₂, Ar₃, Ar₄, and Z in Formula (4) are shown in Table 12.

TABLE 9

| | | | | | | | Ar₃ | | Ar₄ | |
|---|---|---|---|---|---|---|---|---|---|---|
| | | | | | | | substituent | | substituent | End |
| | | | Ar₁ | | Ar₂ | | other than | | other than | group |
| | | Ar₁ | substituent | Ar₂ | substituent | Ar₃ | —CH₂OH | Ar₄ | —Z | (—Z) |
| Synthesis Example 1 | Formula | Formula | —CH₃ | Formula | None | Formula | —(F)₂ | Formula | None | —OH |
| | (1) | (5) | | (5) | | (5) | | (5) | | |
| Synthesis Example 2 | Formula | Formula | —CH₃ | Formula | None | Formula | —F | Formula | None | —OH |
| | (1) | (5) | | (5) | | (5) | | (5) | | |
| Synthesis Example 3 | Formula | Formula | —CH₃ | Formula | None | Formula | —Br | Formula | None | —OH |
| | (1) | (5) | | (5) | | (5) | | (5) | | |
| Synthesis Example 4 | Formula | Formula | —CH₃ | Formula | None | Formula | —NO₂ | Formula | None | —OH |
| | (1) | (5) | | (5) | | (5) | | (5) | | |
| Synthesis Example 5 | Formula | Formula | —CH₃ | Formula | None | Formula | —CH₃ | Formula | None | —OH |
| | (1) | (5) | | (5) | | (5) | | (5) | | |
| Synthesis Example 6 | Formula | Formula | —CH₃ | Formula | None | Formula | —(CH₃)₂ | Formula | None | —OH |
| | (1) | (5) | | (5) | | (5) | | (5) | | |
| Synthesis Example 7 | Formula | Formula | —CH₃ | Formula | None | Formula | —Cl | Formula | None | —OH |
| | (1) | (5) | | (5) | | (5) | | (5) | | |
| Synthesis Example 8 | Formula | Formula | —CH₃ | Formula | None | Formula | —(F)₂ | Formula | None | —OH |
| | (1) | (5) | | (5) | | (5) | | (5) | | |
| Synthesis Example 9 | Formula | Formula | —CH₃ | Formula | None | Formula | —(CH₃)₂ | Formula | None | —OH |
| | (1) | (5) | | (5) | | (5) | | (5) | | |
| Synthesis Example 10 | Formula | Formula | —CH₃ | Formula | None | Formula | —CH₃ | Formula | None | —OH |
| | (1) | (5) | | (5) | | (5) | | (5) | | |
| Synthesis Example 11 | Formula | Formula | —CH₃ | Formula | None | Formula | —NO₂ | Formula | None | —OH |
| | (1) | (5) | | (5) | | (5) | | (5) | | |
| Synthesis Example 12 | Formula | Formula | —CH₃ | Formula | None | Formula | —CF₃ | Formula | None | —OH |
| | (1) | (5) | | (5) | | (5) | | (5) | | |
| Synthesis Example 13 | Formula | Formula | —CH₃ | Formula | None | Formula | —(CH₃)₂ | Formula | None | —OH |
| | (1) | (5) | | (5) | | (5) | | (5) | | |
| Synthesis Example 14 | Formula | Formula | —CH₃ | Formula | None | Formula | —CH₃ | Formula | —F | —OH |
| | (1) | (5) | | (5) | | (5) | | (5) | | |
| Synthesis Example 15 | Formula | Formula | —CH₃ | Formula | None | Formula | —CH₃ | Formula | —(F)₄ | —OH |
| | (1) | (5) | | (5) | | (5) | | (5) | | |
| Synthesis Example 16 | Formula | Formula | —CH₃ | Formula | None | Formula | —CH₃ | Formula | —(F)₂ | —OH |
| | (1) | (5) | | (5) | | (5) | | (5) | | |
| Synthesis Example 17 | Formula | Formula | —CH₃ | Formula | None | Formula | —CH₃ | Formula | None | —COOH |
| | (1) | (5) | | (5) | | (5) | | (5) | | |
| Synthesis Example 18 | Formula | Formula | —CH₃ | Formula | None | Formula | —CH₃ | Formula | None | —CONH₂ |
| | (1) | (5) | | (5) | | (5) | | (5) | | |
| Synthesis Example 19 | Formula | Formula | —CH₃ | Formula | None | Formula | —CH₃ | Formula | None | —NH₂ |
| | (1) | (5) | | (5) | | (5) | | (5) | | |

TABLE 10

| | | | | | | | Ar₃ | | Ar₄ | |
|---|---|---|---|---|---|---|---|---|---|---|
| | | | | | | | substituent | | substituent | End |
| | | | Ar₁ | | Ar₂ | | other than | | other than | group |
| | | Ar₁ | substituent | Ar₂ | substituent | Ar₃ | —CH₂OH | Ar₄ | —Z | (—Z) |
| Synthesis Example 20 | Formula | Formula | —CH₃ | Formula | None | Formula | —CN | Formula | None | —COOH |
| | (2) | (5) | | (5) | | (5) | | (5) | | |
| Synthesis Example 21 | Formula | Formula | —CH₃ | Formula | None | Formula | —CN | Formula | None | —CONH₂ |
| | (2) | (5) | | (5) | | (5) | | (5) | | |
| Synthesis Example 22 | Formula | Formula | —CH₃ | Formula | None | Formula | —CN | Formula | —F | —COOH |
| | (2) | (5) | | (5) | | (5) | | (5) | | |
| Synthesis Example 23 | Formula | Formula | —CH₃ | Formula | None | Formula | —CN | Formula | —Br | —COOH |
| | (2) | (5) | | (5) | | (5) | | (5) | | |
| Synthesis Example 24 | Formula | Formula | —CH₃ | Formula | None | Formula | —CN | Formula | —Cl | —COOH |
| | (2) | (5) | | (5) | | (5) | | (5) | | |
| Synthesis Example 25 | Formula | Formula | —CH₃ | Formula | None | Formula | —CN | Formula | —(F)₂ | —COOH |
| | (2) | (5) | | (5) | | (5) | | (5) | | |
| Synthesis Example 26 | Formula | Formula | —CH₃ | Formula | None | Formula | —CN | Formula | —CH₃ | —COOH |
| | (2) | (5) | | (5) | | (5) | | (5) | | |
| Synthesis Example 27 | Formula | Formula | —CH₃ | Formula | None | Formula | —CN | Formula | —NO₂ | —COOH |
| | (2) | (5) | | (5) | | (5) | | (5) | | |
| Synthesis Example 28 | Formula | Formula | —CH₃ | Formula | None | Formula | —CN | Formula | —CN, —Cl | —COOH |
| | (2) | (5) | | (5) | | (5) | | (5) | | |
| Synthesis Example 29 | Formula | Formula | —CH₃ | Formula | None | Formula | —CN | Formula | —(F)₂ | —COOH |
| | (2) | (5) | | (5) | | (5) | | (5) | | |
| Synthesis Example 30 | Formula | Formula | —CH₃ | Formula | None | Formula | —CN | Formula | —CH₃ | —COOH |
| | (2) | (5) | | (5) | | (5) | | (5) | | |
| Synthesis Example 31 | Formula | Formula | —CH₃ | Formula | None | Formula | —CN | Formula | —(F)₂ | —COOH |
| | (2) | (5) | | (5) | | (5) | | (5) | | |
| Synthesis Example 32 | Formula | Formula | —CH₃ | Formula | None | Formula | —CN | Formula | —CN | —COOH |
| | (2) | (5) | | (5) | | (5) | | (5) | | |
| Synthesis Example 33 | Formula | Formula | —CH₃ | Formula | None | Formula | —CN | Formula | —Br, —F | —COOH |
| | (2) | (5) | | (5) | | (5) | | (5) | | |
| Synthesis Example 34 | Formula | Formula | —CH₃ | Formula | None | Formula | —CN | Formula | —NO₂ | —COOH |
| | (2) | (5) | | (5) | | (5) | | (5) | | |
| Synthesis Example 35 | Formula | Formula | —CH₃ | Formula | None | Formula | —CN | Formula | —(F)₂ | —COOH |
| | (2) | (5) | | (5) | | (5) | | (5) | | |
| Synthesis Example 36 | Formula | Formula | —CH₃ | Formula | None | Formula | —CN | Formula | —(F)₂ | —COOH |
| | (2) | (5) | | (5) | | (5) | | (5) | | |
| Synthesis Example 37 | Formula | Formula | —CH₃ | Formula | None | Formula | —CN | Formula | None | —NH₂ |
| | (2) | (5) | | (5) | | (5) | | (5) | | |
| Synthesis Example 38 | Formula | Formula | —CH₃ | Formula | None | Formula | —CN | Formula | —CF₃ | —NH₂ |
| | (2) | (5) | | (5) | | (5) | | (5) | | |
| Synthesis Example 39 | Formula | Formula | —CH₃ | Formula | None | Formula | —CN | Formula | —CH₃ | —NH₂ |
| | (2) | (5) | | (5) | | (5) | | (5) | | |

TABLE 11

| | | | | | | | Ar₃ | | Ar₄ | |
|---|---|---|---|---|---|---|---|---|---|---|
| | | | | | | | substituent | | substituent | End |
| | | | Ar₁ | | Ar₂ | | other than | | other than | group |
| | | Ar₁ | substituent | Ar₂ | substituent | Ar₃ | —CH₂OH | Ar₄ | —Z | (—Z) |
| Synthesis Example 40 | Formula | Formula | —CH₃ | Formula | None | Formula | None | Formula | —CH₃ | —OH |
| | (3) | (5) | | (5) | | (5) | | (5) | | |
| Synthesis Example 41 | Formula | Formula | None | Formula | None | Formula | None | Formula | None | —OH |
| | (3) | (5) | | (5) | | (5) | | (5) | | |
| Synthesis Example 42 | Formula | Formula | None | Formula | —CH₃ | Formula | —CH₃ | Formula | None | —OH |
| | (3) | (5) | | (5) | | (5) | | (5) | | |
| Synthesis Example 43 | Formula | Formula | —CH₃ | Formula | —CH₃ | Formula | —CH₃ | Formula | —CH₃ | —OH |
| | (3) | (5) | | (5) | | (5) | | (5) | | |
| Synthesis Example 44 | Formula | Formula | —(CH₃)₄ | Formula | None | Formula | None | Formula | —(CH₃)₄ | —OH |
| | (3) | (5) | | (5) | | (5) | | (5) | | |
| Synthesis Example 45 | Formula | Formula | None | Formula | —(CH₃)₄ | Formula | —(CH₃)₄ | Formula | None | —OH |
| | (3) | (5) | | (5) | | (5) | | (5) | | |
| Synthesis Example 46 | Formula | Formula | —(CH₃)₄ | Formula | —(CH₃)₄ | Formula | —(CH₃)₄ | Formula | —(CH₃)₄ | —OH |
| | (3) | (5) | | (5) | | (5) | | (5) | | |
| Synthesis Example 47 | Formula | Formula | —(CH₃)₃ | Formula | None | Formula | None | Formula | —(CH₃)₃ | —OH |
| | (3) | (5) | | (5) | | (5) | | (5) | | |
| Synthesis Example 48 | Formula | Formula | —CH₃ | Formula | None | Formula | None | Formula | —CF₃ | —OH |
| | (3) | (5) | | (5) | | (5) | | (5) | | |
| Synthesis Example 49 | Formula | Formula | —F | Formula | None | Formula | None | Formula | —F | —OH |
| | (3) | (5) | | (5) | | (5) | | (5) | | |
| Synthesis Example 50 | Formula | Formula | —Cl | Formula | None | Formula | None | Formula | —Cl | —OH |
| | (3) | (5) | | (5) | | (5) | | (5) | | |
| Synthesis Example 51 | Formula | Formula | —Br | Formula | None | Formula | None | Formula | —Br | —OH |
| | (3) | (5) | | (5) | | (5) | | (5) | | |
| Synthesis Example 52 | Formula | Formula | —NO₂ | Formula | None | Formula | None | Formula | —NO₂ | —OH |
| | (3) | (5) | | (5) | | (5) | | (5) | | |
| Synthesis Example 53 | Formula | Formula | —CH₃ | Formula | —F | Formula | —F | Formula | —CH₃ | —OH |
| | (3) | (5) | | (5) | | (5) | | (5) | | |
| Synthesis Example 54 | Formula | Formula | —CH₃ | Formula | —Cl | Formula | —Cl | Formula | —CH₃ | —OH |
| | (3) | (5) | | (5) | | (5) | | (5) | | |
| Synthesis Example 55 | Formula | Formula | —CH₃ | Formula | —Br | Formula | —Br | Formula | —CH₃ | —OH |
| | (3) | (5) | | (5) | | (5) | | (5) | | |
| Synthesis Example 56 | Formula | Formula | —CH₃ | Formula | —NO₂ | Formula | —NO₂ | Formula | —CH₃ | —OH |
| | (3) | (5) | | (5) | | (5) | | (5) | | |
| Synthesis Example 57 | Formula | Formula | None | Formula | None | Formula | None | Formula | None | —OH |
| | (3) | (7) | | (5) | | (5) | | (7) | | |
| Synthesis Example 58 | Formula | Formula | None | Formula | None | Formula | None | Formula | None | —OH |
| | (3) | (6) | | (5) | | (5) | | (6) | | |
| Synthesis Example 59 | Formula | Formula | —(CH₃)₄ | Formula | None | Formula | None | Formula | —(CH₃)₄ | —OH |
| | (3) | (9) | | (5) | | (5) | | (9) | | |
| Synthesis Example 60 | Formula | Formula | None | Formula | None | Formula | None | Formula | None | —OH |
| | (3) | (9) | | (5) | | (5) | | (9) | | |
| Synthesis Example 61 | Formula | Formula | —CH₃ | Formula | None | Formula | None | Formula | —CH₃ | —OH |
| | (3) | (5) | | (7) | | (7) | | (5) | | |
| Synthesis Example 62 | Formula | Formula | —CH₃ | Formula | None | Formula | None | Formula | —CH₃ | —OH |
| | (3) | (5) | | (6) | | (6) | | (5) | | |
| Synthesis Example 63 | Formula | Formula | —CH₃ | Formula | None | Formula | None | Formula | —CH₃ | —OH |
| | (3) | (5) | | (9) | | (9) | | (5) | | |
| Synthesis Example 64 | Formula | Formula | None | Formula | None | Formula | None | Formula | None | —OH |
| | (3) | (6) | | (9) | | (9) | | (6) | | |
| Synthesis Example 65 | Formula | Formula | —CH₃ | Formula | None | Formula | None | Formula | —CH₃ | —OH |
| | (3) | (5) | | (5) | | (5) | | (5) | | |
| Synthesis Example 66 | Formula | Formula | —CH₃ | Formula | None | Formula | None | Formula | —CH₃ | —OH |
| | (3) | (5) | | (5) | | (5) | | (5) | | |
| Synthesis Example 67 | Formula | Formula | —CH₃ | Formula | None | Formula | None | Formula | —CH₃ | —OH |
| | (3) | (5) | | (5) | | (5) | | (5) | | |
| Synthesis Example 68 | Formula | Formula | —CH₃ | Formula | None | Formula | None | Formula | —CH₃ | —OH |
| | (3) | (5) | | (8) | | (5) | | (5) | | |

TABLE 12

| | | | | | | | Ar₃ | | Ar₄ | |
|---|---|---|---|---|---|---|---|---|---|---|
| | | | | | | | substituent | | substituent | End |
| | | | Ar₁ | | Ar₂ | | other than | | other than | group |
| | | Ar₁ | substituent | Ar₂ | substituent | Ar₃ | —CH₂OH | Ar₄ | —Z | (—Z) |
| Synthesis Example 69 | Formula | Formula | —CH₃ | Formula | None | Formula | None | Formula | None | —OH |
| | (4) | (5) | | (5) | | (5) | | (5) | | |
| Synthesis Example 70 | Formula | Formula | —CH₃ | Formula | None | Formula | None | Formula | —F | —OH |
| | (4) | (5) | | (5) | | (5) | | (5) | | |
| Synthesis Example 71 | Formula | Formula | —CH₃ | Formula | None | Formula | None | Formula | —(F)₄ | —OH |
| | (4) | (5) | | (5) | | (5) | | (5) | | |
| Synthesis Example 72 | Formula | Formula | —CH₃ | Formula | None | Formula | None | Formula | —(F)₂ | —OH |
| | (4) | (5) | | (5) | | (5) | | (5) | | |
| Synthesis Example 73 | Formula | Formula | —CH₃ | Formula | None | Formula | None | Formula | None | —COOH |
| | (4) | (5) | | (5) | | (5) | | (5) | | |
| Synthesis Example 74 | Formula | Formula | —CH₃ | Formula | None | Formula | None | Formula | None | —CONH₂ |
| | (4) | (5) | | (5) | | (5) | | (5) | | |
| Synthesis Example 75 | Formula | Formula | —CH₃ | Formula | None | Formula | None | Formula | None | —NH₂ |
| | (4) | (5) | | (5) | | (5) | | (5) | | |

### <Production of Resin Composition>

### Examples 1 to 83

The epoxy resins shown in Tables 13 to 16, the compounds shown in Tables 13 to 16 and serving as curing agents, and the curing accelerators shown in Tables 13 to 16 were mixed in the ratios shown in Tables 13 to 16, respectively, to obtain resin compositions of Examples 1 to 83.

TABLE 13

| | | | | | | Curing | Thermal |
|---|---|---|---|---|---|---|---|
| | | | Curing | Compound | Epoxy resin | accelerator | conductivity |
| | Curing agent | Epoxy resin | accelerator | (% by mass) | (% by mass) | (% by mass) | W/(m · K) |
| Example 1 | Synthesis Example 1 | TEPIC-SS | 2E4MZ | 85 | 12 | 3 | 0.45 |
| Example 2 | Synthesis Example 2 | TEPIC-SS | 2E4MZ | 85 | 12 | 3 | 0.55 |
| Example 3 | Synthesis Example 3 | TEPIC-SS | 2E4MZ | 85 | 12 | 3 | 0.44 |
| Example 4 | Synthesis Example 4 | TEPIC-SS | 2E4MZ | 85 | 12 | 3 | 0.47 |
| Example 5 | Synthesis Example 5 | TEPIC-SS | 2E4MZ | 85 | 12 | 3 | 0.52 |
| Example 6 | Synthesis Example 6 | TEPIC-SS | 2E4MZ | 85 | 12 | 3 | 0.50 |
| Example 7 | Synthesis Example 7 | TEPIC-SS | 2E4MZ | 85 | 12 | 3 | 0.56 |
| Example 8 | Synthesis Example 8 | TEPIC-SS | 2E4MZ | 85 | 12 | 3 | 0.54 |
| Example 9 | Synthesis Example 9 | TEPIC-SS | 2E4MZ | 85 | 12 | 3 | 0.49 |
| Example 10 | Synthesis Example 10 | TEPIC-SS | 2E4MZ | 85 | 12 | 3 | 0.48 |
| Example 11 | Synthesis Example 11 | TEPIC-SS | 2E4MZ | 85 | 12 | 3 | 0.53 |
| Example 12 | Synthesis Example 12 | TEPIC-SS | 2E4MZ | 85 | 12 | 3 | 0.55 |
| Example 13 | Synthesis Example 13 | TEPIC-SS | 2E4MZ | 85 | 12 | 3 | 0.49 |
| Example 14 | Synthesis Example 14 | TEPIC-SS | 2E4MZ | 85 | 12 | 3 | 0.45 |
| Example 15 | Synthesis Example 15 | TEPIC-SS | 2E4MZ | 85 | 12 | 3 | 0.48 |
| Example 16 | Synthesis Example 16 | TEPIC-SS | 2E4MZ | 85 | 12 | 3 | 0.56 |
| Example 17 | Synthesis Example 17 | TEPIC-SS | 2E4MZ | 85 | 12 | 3 | 0.54 |
| Example 18 | Synthesis Example 18 | TEPIC-SS | 2E4MZ | 85 | 12 | 3 | 0.54 |
| Example 19 | Synthesis Example 19 | TEPIC-SS | 2E4MZ | 85 | 12 | 3 | 0.54 |
| Example 20 | Synthesis Example 20 | TEPIC-SS | 2E4MZ | 85 | 12 | 3 | 0.54 |
| Example 21 | Synthesis Example 21 | TEPIC-SS | 2E4MZ | 85 | 12 | 3 | 0.53 |

TABLE 14

| | | | | | | Curing | Thermal |
|---|---|---|---|---|---|---|---|
| | | | Curing | Compound | Epoxy resin | accelerator | conductivity |
| | Curing agent | Epoxy resin | accelerator | (% by mass) | (% by mass) | (% by mass) | W/(m · K) |
| Example 22 | Synthesis Example 22 | TEPIC-SS | 2E4MZ | 85 | 12 | 3 | 0.47 |
| Example 23 | Synthesis Example 23 | TEPIC-SS | 2E4MZ | 85 | 12 | 3 | 0.44 |
| Example 24 | Synthesis Example 24 | TEPIC-SS | 2E4MZ | 85 | 12 | 3 | 0.52 |
| Example 25 | Synthesis Example 25 | TEPIC-SS | 2E4MZ | 85 | 12 | 3 | 0.46 |
| Example 26 | Synthesis Example 26 | TEPIC-SS | 2E4MZ | 85 | 12 | 3 | 0.45 |
| Example 27 | Synthesis Example 27 | TEPIC-SS | 2E4MZ | 85 | 12 | 3 | 0.49 |
| Example 28 | Synthesis Example 28 | TEPIC-SS | 2E4MZ | 85 | 12 | 3 | 0.47 |
| Example 29 | Synthesis Example 29 | TEPIC-SS | 2E4MZ | 85 | 12 | 3 | 0.49 |
| Example 30 | Synthesis Example 30 | TEPIC-SS | 2E4MZ | 85 | 12 | 3 | 0.44 |
| Example 31 | Synthesis Example 31 | TEPIC-SS | 2E4MZ | 85 | 12 | 3 | 0.53 |
| Example 32 | Synthesis Example 32 | TEPIC-SS | 2E4MZ | 85 | 12 | 3 | 0.45 |
| Example 33 | Synthesis Example 33 | TEPIC-SS | 2E4MZ | 85 | 12 | 3 | 0.44 |
| Example 34 | Synthesis Example 34 | TEPIC-SS | 2E4MZ | 85 | 12 | 3 | 0.48 |
| Example 35 | Synthesis Example 35 | TEPIC-SS | 2E4MZ | 85 | 12 | 3 | 0.47 |
| Example 36 | Synthesis Example 36 | TEPIC-SS | 2E4MZ | 85 | 12 | 3 | 0.45 |
| Example 37 | Synthesis Example 37 | TEPIC-SS | 2E4MZ | 85 | 12 | 3 | 0.51 |
| Example 38 | Synthesis Example 38 | TEPIC-SS | 2E4MZ | 85 | 12 | 3 | 0.46 |
| Example 39 | Synthesis Example 39 | TEPIC-SS | 2E4MZ | 85 | 12 | 3 | 0.43 |
| Example 40 | Synthesis Example 40 | TEPIC-SS | 2E4MZ | 85 | 12 | 3 | 0.58 |
| Example 41 | Synthesis Example 41 | TEPIC-SS | 2E4MZ | 85 | 12 | 3 | 0.55 |

TABLE 15

| | | | | | | Curing | Thermal |
|---|---|---|---|---|---|---|---|
| | | | Curing | Compound | Epoxy resin | accelerator | conductivity |
| | Curing agent | Epoxy resin | accelerator | (% by mass) | (% by mass) | (% by mass) | W/(m · K) |
| Example 42 | Synthesis Example 42 | TEPIC-SS | 2E4MZ | 85 | 12 | 3 | 0.51 |
| Example 43 | Synthesis Example 43 | TEPIC-SS | 2E4MZ | 85 | 12 | 3 | 0.53 |
| Example 44 | Synthesis Example 44 | TEPIC-SS | 2E4MZ | 85 | 12 | 3 | 0.52 |
| Example 45 | Synthesis Example 45 | TEPIC-SS | 2E4MZ | 85 | 12 | 3 | 0.48 |
| Example 46 | Synthesis Example 46 | TEPIC-SS | 2E4MZ | 85 | 12 | 3 | 0.49 |
| Example 47 | Synthesis Example 47 | TEPIC-SS | 2E4MZ | 85 | 12 | 3 | 0.44 |
| Example 48 | Synthesis Example 48 | TEPIC-SS | 2E4MZ | 85 | 12 | 3 | 0.46 |
| Example 49 | Synthesis Example 49 | TEPIC-SS | 2E4MZ | 85 | 12 | 3 | 0.42 |
| Example 50 | Synthesis Example 50 | TEPIC-SS | 2E4MZ | 85 | 12 | 3 | 0.52 |
| Example 51 | Synthesis Example 51 | TEPIC-SS | 2E4MZ | 85 | 12 | 3 | 0.49 |
| Example 52 | Synthesis Example 52 | TEPIC-SS | 2E4MZ | 85 | 12 | 3 | 0.45 |
| Example 53 | Synthesis Example 53 | TEPIC-SS | 2E4MZ | 85 | 12 | 3 | 0.55 |
| Example 54 | Synthesis Example 54 | TEPIC-SS | 2E4MZ | 85 | 12 | 3 | 0.53 |
| Example 55 | Synthesis Example 55 | TEPIC-SS | 2E4MZ | 85 | 12 | 3 | 0.49 |
| Example 56 | Synthesis Example 56 | TEPIC-SS | 2E4MZ | 85 | 12 | 3 | 0.42 |
| Example 57 | Synthesis Example 57 | TEPIC-SS | 2E4MZ | 85 | 12 | 3 | 0.55 |
| Example 58 | Synthesis Example 58 | TEPIC-SS | 2E4MZ | 85 | 12 | 3 | 0.44 |
| Example 59 | Synthesis Example 59 | TEPIC-SS | 2E4MZ | 85 | 12 | 3 | 0.56 |
| Example 60 | Synthesis Example 60 | TEPIC-SS | 2E4MZ | 85 | 12 | 3 | 0.45 |
| Example 61 | Synthesis Example 61 | TEPIC-SS | 2E4MZ | 85 | 12 | 3 | 0.49 |

TABLE 16

| | | | | | | Curing | Thermal |
|---|---|---|---|---|---|---|---|
| | | | Curing | Compound | Epoxy resin | accelerator | conductivity |
| | Curing agent | Epoxy resin | accelerator | (% by mass) | (% by mass) | (% by mass) | W/(m · K) |
| Example 62 | Synthesis Example 62 | TEPIC-SS | 2E4MZ | 85 | 12 | 3 | 0.50 |
| Example 63 | Synthesis Example 63 | TEPIC-SS | 2E4MZ | 85 | 12 | 3 | 0.52 |
| Example 64 | Synthesis Example 64 | TEPIC-SS | 2E4MZ | 85 | 12 | 3 | 0.52 |
| Example 65 | Synthesis Example 65 | TEPIC-SS | 2E4MZ | 85 | 12 | 3 | 0.51 |
| Example 66 | Synthesis Example 66 | TEPIC-SS | 2E4MZ | 85 | 12 | 3 | 0.47 |
| Example 67 | Synthesis Example 67 | TEPIC-SS | 2E4MZ | 85 | 12 | 3 | 0.50 |
| Example 68 | Synthesis Example 68 | TEPIC-SS | 2E4MZ | 85 | 12 | 3 | 0.41 |
| Example 69 | Synthesis Example 69 | TEPIC-SS | 2E4MZ | 85 | 12 | 3 | 0.56 |
| Example 70 | Synthesis Example 70 | TEPIC-SS | 2E4MZ | 85 | 12 | 3 | 0.47 |
| Example 71 | Synthesis Example 71 | TEPIC-SS | 2E4MZ | 85 | 12 | 3 | 0.49 |
| Example 72 | Synthesis Example 72 | TEPIC-SS | 2E4MZ | 85 | 12 | 3 | 0.43 |
| Example 73 | Synthesis Example 73 | TEPIC-SS | 2E4MZ | 85 | 12 | 3 | 0.53 |
| Example 74 | Synthesis Example 74 | TEPIC-SS | 2E4MZ | 85 | 12 | 3 | 0.52 |
| Example 75 | Synthesis Example 75 | TEPIC-SS | 2E4MZ | 85 | 12 | 3 | 0.51 |
| Example 76 | Synthesis Example 40 | TEPIC-SS | 2E4MZ | 90 | 7 | 3 | 0.56 |
| Example 77 | Synthesis Example 40 | TEPIC-SS | 2E4MZ | 79 | 18 | 3 | 0.54 |
| Example 78 | Synthesis Example 40 | TEPIC-SS | 2E4MZ | 72 | 25 | 3 | 0.49 |
| Example 79 | Synthesis Example 40 | JER630 | 2E4MZ | 85 | 12 | 3 | 0.57 |
| Example 80 | Synthesis Example 40 | EPICLON | 2E4MZ | 85 | 12 | 3 | 0.55 |
| | | HP-4032 | | | | | |
| Example 81 | Synthesis Example 40 | N-655-EXP-S | 2E7MZ | 85 | 12 | 3 | 0.51 |
| Example 82 | Synthesis Example 40 | JER152 | 2E8MZ | 85 | 12 | 3 | 0.53 |
| Example 83 | Synthesis Example 40 | JER828 | 2E9MZ | 85 | 12 | 3 | 0.50 |
| Example 84 | Synthesis Example | TEPIC-SS | 2E4MZ | 85 | 12 | 3 | 0.50 |
| | 40:Compound (A) | | | | | | |
| | (1:1) | | | | | | |

### Examples 84

A curing agent, obtained by mixing the compound of Synthesis Example 40 with the compound (number-average molecular weight (Mn) of 1,728 and weight-average molecular weight (Mw) of 3,300) represented by Formula (A) in a mass ratio of 1:1, was used, and the epoxy resin shown in Table 16 and the curing accelerator shown in Table 16 were mixed in a ratio shown in Table 16 to obtain a resin composition of Example 84.

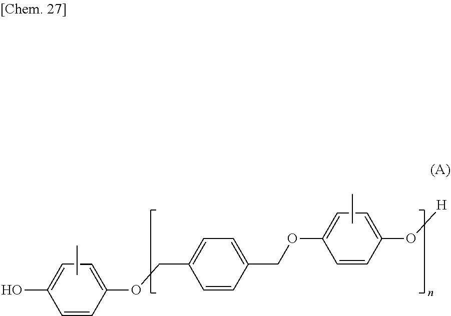

The epoxy resins and curing accelerators shown in Tables 13 to 17 are the following compounds.

### “Epoxy Resin”

TEPIC-SS; Triglycidyl isocyanurate (produced by Nissan Chemical Corporation)
JER630; Triglycidyl-p-aminophenol (produced by Mitsubishi Chemical Corporation)
EPICLON HP-4032; 1,6-Bis(2,3-epoxypropan-1-yloxy)naphthalene (produced by DIC CORPORATION)
N-655-EXP-S; Cresol novolac epoxy resin (produced by DIC CORPORATION) JER152; Cresol novolac epoxy resin represented by Formula (B-1) (produced by Mitsubishi Chemical Corporation)
JER828; Bisphenol A-type epoxy resin (produced by Mitsubishi Chemical Corporation)

### “Curing Accelerator”

2E4MZ; 2-Ethyl-4-methylimidazole

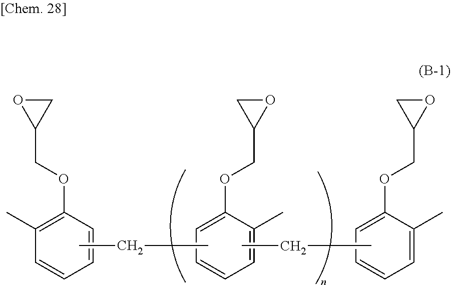

### Examples 85 to 93

The curing agents, obtained by mixing the compound of Synthesis Example 40 or 75 with the compound (number-average molecular weight (Mn) of 1,728 and weight-average molecular weight (Mw) of 3,300) represented by Formula (A) in a mass ratio of 1:1, were used, and the bismaleimide compounds shown in Table 17 and the curing accelerators shown in Table 17 were mixed in ratios shown in Table 17, respectively, to obtain resin compositions of Examples 85 to 93.

Specifically, the curing agents and bismaleimide compounds shown in Table 17 were heated to 150° C., and melted and mixed. The curing accelerators were added thereto and mixed quickly. Thereafter, the obtained mixtures were reacted at 150° C. for 1 hour and at 180° C. for another 1 hour to prepare cured products (resin compositions of Examples 85 to 93).

TABLE 17

| | | | | | Bismaleimide | Curing | Thermal |
|---|---|---|---|---|---|---|---|
| | | | Curing | Compound | resin | accelerator | conductivity |
| | Curing agent | Bismaleimide compound | accelerator | (% by mass) | (% by mass) | (% by mass) | W/(m · K) |
| Example 85 | Synthesis Example | 1,2-Bis(maleimido)ethane | 2E4MZ | 87 | 10 | 3 | 0.60 |
| | 40:Compound (A) | | | | | | |
| | (1:1) | | | | | | |
| Example 86 | Synthesis Example | 1,4-Bis(maleimido)butane | 2E4MZ | 86 | 11 | 3 | 0.57 |
| | 40:Compound (A) | | | | | | |
| | (1:1) | | | | | | |
| Example 87 | Synthesis Example | 1.6-Bis(maleimido)hexane | 2E4MZ | 84 | 13 | 3 | 0.55 |
| | 40:Compound (A) | | | | | | |
| | (1:1) | | | | | | |
| Example 88 | Synthesis Example | N,N′-1,4-Phenylenedimaleimide | 2E4MZ | 85 | 12 | 3 | 0.59 |
| | 40:Compound (A) | | | | | | |
| | (1:1) | | | | | | |
| Example 89 | Synthesis Example | N,N′-1,3-Phenylenedimaleimide | 2E4MZ | 85 | 12 | 3 | 0.51 |
| | 40:Compound (A) | | | | | | |
| | (1:1) | | | | | | |
| Example 90 | Synthesis Example | 4,4′-Bismaleimidodiphenylmethane | 2E4MZ | 81 | 16 | 3 | 0.46 |
| | 40:Compound (A) | | | | | | |
| | (1:1) | | | | | | |
| Example 91 | Synthesis Example | Bis(3-ethyl-5-methyl-4- | 2E4MZ | 77 | 20 | 3 | 0.44 |
| | 40:Compound (A) | maleimidophenyl)methane | | | | | |
| | (1:1) | | | | | | |
| Example 92 | Synthesis Example | 1,2-Bis(maleimido)ethane | 2E4MZ | 87 | 10 | 3 | 0.58 |
| | 75:Compound (A) | | | | | | |
| | (1:1) | | | | | | |
| Example 93 | Synthesis Example | N,N′-1,4-Phenylenedimaleimide | 2E4MZ | 85 | 12 | 3 | 0.57 |
| | 75:Compound (A) | | | | | | |
| | (1:1) | | | | | | |

The thermal conductivity of each of the resin compositions of Examples 1 to 93 obtained as described above was determined by the following method. The results thereof are shown in Tables 13 to 17.

### (Measurement of Thermal Conductivity)

The density, specific heat, and thermal diffusivity of the resin cured product were measured by the following methods, respectively, and the thermal conductivity was determined by multiplying them.

The density was determined using the Archimedes method.

The specific heat was determined using a differential scanning calorimeter (DSC) (manufactured by Hitachi High-Tech Science Corporation).

The thermal diffusivity was obtained using a xenon flash thermal diffusivity measurement device (ADVANCE RIKO, Inc.).

A measurement sample produced by the following method was used for the measurement of the thermal diffusivity. That is, the resin cured product was melted and mixed at a temperature of 150° C. in an aluminum cup, and cooled to room temperature. Thereafter, the uncured resin composition was cured by heating at 150° C. for 12 hours. The obtained resin cured product was processed into a cylindrical shape having a diameter of 10 mm and a thickness of 0.5 mm, and the resultant was used as the measurement sample.

As shown in Tables 13 to 17, the cured products of the resin compositions of Examples 1 to 93 all had thermal conductivity of 0.4 W/(m·K) or greater, indicating high thermal conductivity.

### INDUSTRIAL APPLICABILITY

A cured product having higher thermal conductivity can be obtained.

### REFERENCE SIGNS LIST

10 Resin substrate
12 Resin sheet
20 Cured product
22 Resin component
30 Core material
50 Laminated substrate

1. A compound having a chain structure consisting of an aromatic ring group, an ether oxygen atom, a methylene group, an aromatic ring group, a methylene group, an ether oxygen atom, and an aromatic ring group bonded in an order,
wherein a hydroxymethyl group is bonded to a carbon atom of the aromatic ring group disposed at a first end of the chain structure, and
one end group selected from a hydroxyl group, an amino group, an amide group, and a carboxyl group is bonded to a carbon atom of the aromatic ring group disposed at a second end of the chain structure.

2. The compound according to claim 1, comprising:
a first aromatic ring unit composed of a first aromatic ring group and two ether oxygen atoms bonded to the first aromatic ring group;
a second aromatic ring unit composed of a second aromatic ring group and two methylene groups bonded to the second aromatic ring group;
a third aromatic ring unit composed of a third aromatic ring group and a hydroxymethyl group bonded to the third aromatic ring group; and
a fourth aromatic ring unit composed of a fourth aromatic ring group and the end group bonded to the fourth aromatic ring group,
wherein the chain structure has a skeleton in which the first aromatic ring unit and the second aromatic ring unit are alternately disposed and the first aromatic ring unit is disposed at both ends, the third aromatic ring group is bonded to a first end of the skeleton via a methylene group, and the fourth aromatic ring group is bonded to a second end of the skeleton via a methylene group, or
the chain structure has a skeleton in which the first aromatic ring unit and the second aromatic ring unit are alternately disposed and the second aromatic ring unit is disposed at both ends, the third aromatic ring group is bonded to a first end of the skeleton via an ether oxygen atom, and the fourth aromatic ring group is bonded to a second end of the skeleton via an ether oxygen atom.

3. The compound according to claim 1, comprising:
a first aromatic ring unit composed of a first aromatic ring group and two ether oxygen atoms bonded to the first aromatic ring group;
a second aromatic ring unit composed of a second aromatic ring group and two methylene groups bonded to the second aromatic ring group;
a third aromatic ring unit composed of a third aromatic ring group and a hydroxymethyl group bonded to the third aromatic ring group; and
a fourth aromatic ring unit composed of a fourth aromatic ring group and the end group bonded to the fourth aromatic ring group,
wherein the chain structure has a skeleton in which the first aromatic ring unit and the second aromatic ring unit are alternately disposed, and
the third aromatic ring group is bonded to an end on the first aromatic ring unit side via a methylene group, and the fourth aromatic ring group is bonded to an end on the second aromatic ring unit side via an ether oxygen atom, or
the fourth aromatic ring group is bonded to an end on the first aromatic ring unit side via a methylene group, and the third aromatic ring group is bonded to an end on the second aromatic ring unit side via an ether oxygen atom.

4. The compound according to claim 1, which is represented by one of General Formulae (1) to (4): 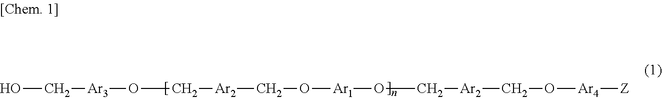
(in Formula (1), Ar₁'s are each independently a first aromatic ring group which may have a substituent, Ar₂'s are each independently a second aromatic ring group which may have a substituent, Ar₃ is a third aromatic ring group which may have a substituent, Ar₄ is a fourth aromatic ring group which may have a substituent, Z is the end group, and n is an integer of 0 or more); 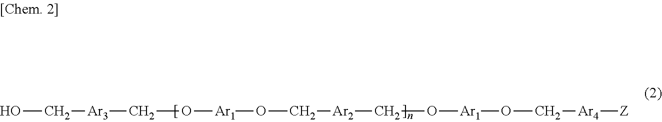
(in Formula (2), Ar₁'s are each independently a first aromatic ring group which may have a substituent, Ar₂'s are each independently a second aromatic ring group which may have a substituent, Ar₃ is a third aromatic ring group which may have a substituent, Ar₄ is a fourth aromatic ring group which may have a substituent, Z is the end group, and n is an integer of 1 or more); 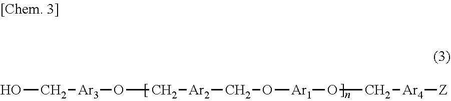
(in Formula (3), Ar₁'s are each independently a first aromatic ring group which may have a substituent, Ar₂'s are each independently a second aromatic ring group which may have a substituent, Ar₃ is a third aromatic ring group which may have a substituent, Ar₄ is a fourth aromatic ring group which may have a substituent, Z is the end group, and n is an integer of 1 or more); and 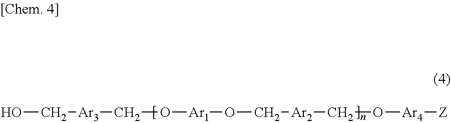
(in Formula (4), Ar₁'s are each independently a first aromatic ring group which may have a substituent, Ar₂'s are each independently a second aromatic ring group which may have a substituent, Ar₃ is a third aromatic ring group which may have a substituent, Ar₄ is a fourth aromatic ring group which may have a substituent, Z is the end group, and n is an integer of 1 or more).

5. The compound according to claim 1,
wherein one or more of the first aromatic ring group, the second aromatic ring group, the third aromatic ring group, and the fourth aromatic ring group are any aromatic ring groups represented by Formulae (5) to (9): 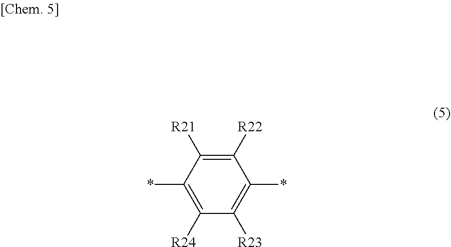
(in Formula (5), R21 to R24 are each independently one selected from hydrogen, a methyl group, a trifluoromethyl group, a halogen group, a nitro group, and a cyano group, and * is a bond); 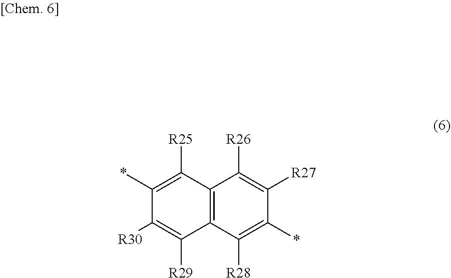
(in Formula (6), R25 to R30 are each independently one selected from hydrogen, a methyl group, a trifluoromethyl group, a halogen group, a nitro group, and a cyano group, and * is a bond); 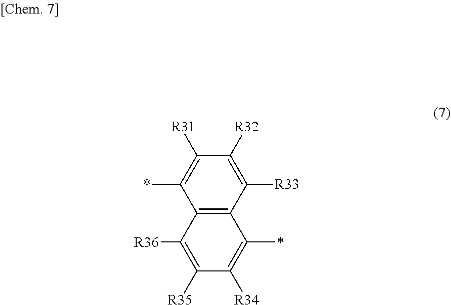
(in Formula (7), R31 to R36 are each independently one selected from hydrogen, a methyl group, a trifluoromethyl group, a halogen group, a nitro group, and a cyano group, and * is a bond); 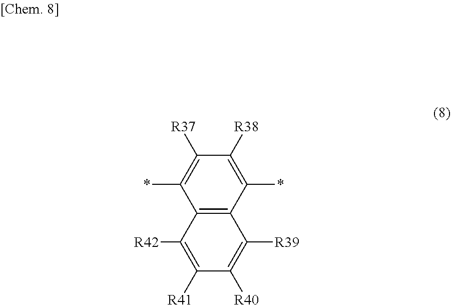
(in Formula (8), R37 to R42 are each independently one selected from hydrogen, a methyl group, a trifluoromethyl group, a halogen group, a nitro group, and a cyano group, and * is a bond); and 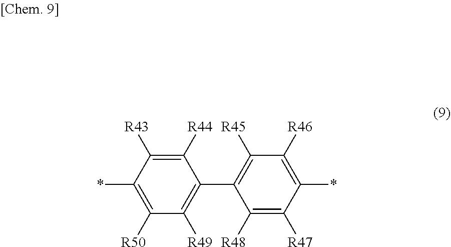
(in Formula (9), R43 to R50 are each independently one selected from hydrogen, a methyl group, a trifluoromethyl group, a halogen group, a nitro group, and a cyano group, and * is a bond).

6. The compound according to claim 1,
wherein one or more of the first aromatic ring group, the second aromatic ring group, the third aromatic ring group, and the fourth aromatic ring group are paraphenylene groups which may have a substituent.

7. The compound according to claim 2,
wherein the first aromatic ring group and the fourth aromatic ring group are a same, and
the second aromatic ring group is a paraphenylene group.

8. The compound according to claim 1, which is represented by one of General Formulae (10) to (13): 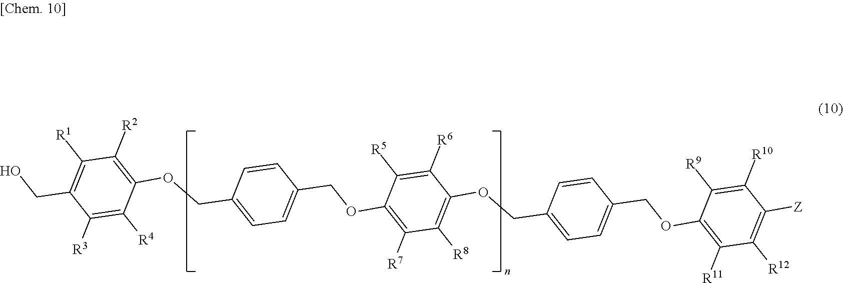
(in Formula (10), R1 to R12 are each independently one selected from hydrogen, a methyl group, a trifluoromethyl group, a halogen group, a nitro group, and a cyano group, Z is the end group, and n is an integer of 1 or more); 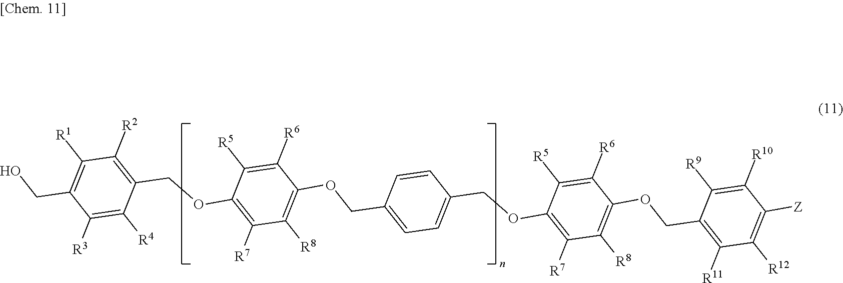
(in Formula (11), R1 to R12 are each independently one selected from hydrogen, a methyl group, a trifluoromethyl group, a halogen group, a nitro group, and a cyano group, Z is the end group, and n is an integer of 1 or more); 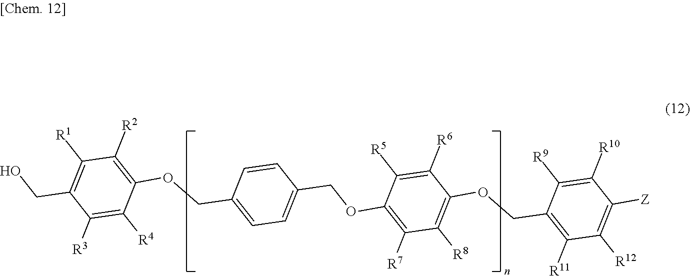
(in Formula (12), R1 to R12 are each independently one selected from hydrogen, a methyl group, a trifluoromethyl group, a halogen group, a nitro group, and a cyano group, Z is the end group, and n is an integer of 1 or more); and 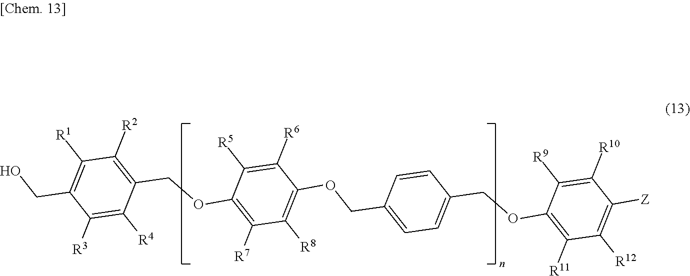
(in Formula (13), R1 to R12 are each independently one selected from hydrogen, a methyl group, a trifluoromethyl group, a halogen group, a nitro group, and a cyano group, Z is the end group, and n is an integer of 1 or more).

9. The compound according to claim 8, which is the compound represented by General Formula (13),
wherein R1 to R4 are each hydrogen,
one among R5 to R8 is a methyl group, and the others are each hydrogen, and
one among R9 to R12 is a methyl group, and the others are each hydrogen.

10. A resin composition comprising the compound according to claim 1.

11. The resin composition according to claim 10, which has a chain structure consisting of an aromatic ring group, an ether oxygen atom, a methylene group, an aromatic ring group, a methylene group, an ether oxygen atom, and an aromatic ring group bonded in an order, the resin composition comprising one or both of a compound in which one end group selected from a hydroxyl group, an amino group, an amide group, and a carboxyl group is bonded to a carbon atom of each of the aromatic ring groups disposed at both ends of the chain structure, and a compound in which a hydroxymethyl group is bonded to a carbon atom of each of the aromatic ring groups disposed at both ends of the chain structure.

12. A resin composition comprising an epoxy resin and a curing agent,
wherein the curing agent includes the compound according to claim 1.

13. A resin sheet comprising the resin composition according to claim 1.

14. A resin cured product which includes a cured product of the resin composition according to claim 1.

15. A laminated substrate in which a plurality of resin substrates are laminated,
wherein at least one among the plurality of resin substrates is the resin cured product according to claim 14.
